SEQUENCE LISTING (12 sequences)

SEQ ID NO: 1 - protein, 153 aa, Human immunodeficiency virus
AVGIGALFLGFLGARQLLSGIVQQQNNLLRAIEAQQHLLQLTVWGIKQLQARILAVERYLKDQQDLGIEGCSGKGDCTPEVPWNASDSNKSLEQIWNNMTWMEWDREINNYTSLIHSLIEESQNQQEKNEQELLELDKWASLWNWFNITNWLW

SEQ ID NO: 2 - protein, 11 aa, Human immunodeficiency virus
MHKVHGSGSGS

SEQ ID NO: 3 - protein, 16 aa, Human immunodeficiency virus
AGSTMGARSMTLTVQA

SEQ ID NO: 4 - protein, 7 aa, Human immunodeficiency virus
ELDKWAS

SEQ ID NO: 5 - protein, 6 aa, Human immunodeficiency virus
NWFNIT

SEQ ID NO: 6 - protein, 4 aa, Human immunodeficiency virus
NWLW

SEQ ID NO: 7 - DNA, 33 bp, Human immunodeficiency virus
atgcataaagttcatggtagcggtagcggcagc

SEQ ID NO: 8 - protein, 13 aa, Human immunodeficiency virus
AVGIGALFLGFLG

SEQ ID NO: 9 - protein, 12 aa, Human immunodeficiency virus
LWNWFNITNWLW

SEQ ID NO: 10 - protein, 345 aa, Human immunodeficiency virus
AVGIGALFLGFLGAAGSTMGARSMTLTVQARQLLSGIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKIFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 11 - protein, 164 aa, Human immunodeficiency virus
MHKVHGSGSGSAVGIGALFLGFLGARQLLSGIVQQQNNLLRAIEAQQHLLQLTVWGIKQLQARILAVERYLKDQQDLGIEGCSGKGDCTPEVPWNASDSNKSLEQIWNNMTWMEWDREINNYTSLIHSLIEESQNQQEKNEQELLELDKWASLWNWFNITNWLW

SEQ ID NO: 12 - protein, 176 aa, Human immunodeficiency virus
YIKIFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

(19) United States
(12) Patent Application Publication
(10) Pub. No.: US 2014/0093556 A1
(43) Pub. Date: Apr. 3, 2014

(54) Immunological Compositions Against HIV

(71) Applicants: Francisco Conejero-Lara, Granada (ES); Irene Luque, Granada (ES); Pedro Luis Mateo, Granada (ES); Andreas Wagner, Wien (AT); Raphaelle Claude, Chaponost (FR); Marie-Gaelle Roger, Proveysieux (FR); Nicolas Mouz, Saint Paul de Varces (FR); Christophe Martin, Coulombs (FR)

(72) Inventors: Francisco Conejero-Lara, Granada (ES); Irene Luque, Granada (ES); Pedro Luis Mateo, Granada (ES); Andreas Wagner, Wien (AT); Raphaelle Claude, Chaponost (FR); Marie-Gaelle Roger, Proveysieux (FR); Nicolas Mouz, Saint Paul de Varces (FR); Christophe Martin, Coulombs (FR)

(73) Assignees: SANOFI PASTEUR SA, Lyon (FR); POLYMUNE SCIENTIFIC IMMUNOBIOLOGISCHE FORSCHUNG GMBH, Klostemeuburg (AT); UNIVERSIDAD DE GRANADA, Grenada (ES); PX' THERAPEUTICS, Grenoble (FR)

(21) Appl. No.: 13/981,671

(22) PCT Filed: Jan. 26, 2012
(86) PCT No.: PCT/IB12/00118

§ 371 Date: Nov. 5, 2013

Related U.S. Application Data
(60) Provisional application No. 61/437,514, filed on Jan. 28, 2011.
(60) Provisional application No. 61/454,693, filed on Mar. 21, 2011.

(51) Int. Cl. C07K 14/005

(52) U.S. Cl. CPC C07K 14/005
USPC 424/450; 424/188.1; 424/208.1; 435/69.3; 435/252.33; 530/350; 530/387.9; 536/23.72

(57) ABSTRACT

The disclosure relates to immunological compositions for vaccinating human beings against infection by the Human Immunodeficiency Virus (HIV).

### RELATED APPLICATIONS

This application claims priority to U.S. Ser. No. 61/437,514 filed Jan. 28, 2011 and U.S. Ser. No. 61/454,693 filed Mar. 21, 2011.

### FIELD OF THE DISCLOSURE

This disclosure relates generally to the field of immunology and, in particular to methods and compositions for immunizing and generating protection in a host against infection and disease with HIV.

### BACKGROUND OF THE DISCLOSURE

Human immunodeficiency virus (HIV) is a human retrovirus and is the etiological agent of acquired immunodeficiency syndrome (AIDS). Despite the passage of more than 20 years since the discovery of HIV, no effective vaccine has been found to either ameliorate the disease or to prevent infection. By the end of the year 2007, more than 30 million people worldwide were infected with HIV, with more than 20 million of those people living in sub-Saharan Africa (Report on the Global AIDS Epidemic, Joint United Nations Programme on HIV/AIDS (UNAIDS), 2008).

A hallmark of resistance to future viral infection is the generation of ‘neutralizing antibodies’ capable of recognizing the viral pathogen. Another measure is cellular immunity against infected cells. In typical viral infections, generation of neutralizing antibodies and cellular immunity heralds recovery from infection. In HIV-1 infection, however, neutralizing antibodies and cellular immunity appear very early during the infection, usually after a few months and have been associated with only a transient decrease in viral burden. In spite of the generation of neutralizing antibodies and cellular immunity, viral replication in HIV-1 infection rebounds and AIDS (acquired immune deficiency syndrome) develops. Thus, in HIV-1 infection, neutralizing antibodies and cellular immunity are not accurate measures of protective immunity against disease development. However, neutralising Ab are able to prevent infection. This was demonstrated in macaque experimental models where infusion of neutralising Abs was able to protect the animals from a viral challenge (Mascola, et al. protection of macaques against vaginal transmission of a pathogenic HIV-1/SIV chimeric virus by passive infusion of neutralizing antibodies. Nat. Med. 6: 207-210 (2000); Hessel, et al. PLoS-Pathog, vol. 5, no. 5, p. e1000433 (2009)). Protective immunity, meaning the vaccinees are protected against infections by HIV, is a major unaccomplished goal of those skilled in the art.

Several potential vaccines have been tested in humans but found not to be protective. For examples, subunit vaccines based on gp120 have been tested (e.g., AIDSVAX® B/B, AIDSVAX®B/E (Vaxgen)) as solo vaccines, but have not shown protection against HIV infection (McCarthy, M. Lancet. 362(9397):1728 (2003); Nitayaphan, et al. J. Inf. Dis. 190:702-6 (2004); Pitisuttithum, P. 11^{th} Conf. Retr. Opp. Inf. 2004. 115: Abstract 107). Many studies have also been performed using animal models (e.g., monkeys). However, while primate data are instructive they also highlight the gaps in our understanding of immunological mechanism that mediate vaccine associated protection and emphasize the need to conduct human efficacy studies to test promising candidate vaccines empirically.

ALVAC-HIV (vCP1521) vaccine is a preparation of recombinant canarypox-derived virus expressing the products of the HIV-1 env and gag genes. The genes are inserted into the C6 locus under the control of the vaccinia virus H6 and I3L promoters respectively. The gp120 env sequence is derived from the HIV-92TH023 (subtype E) strain, but the anchoring part of gp41 is derived from the HIV-LAI (subtype B) strain. ALVAC-HIV infected cells present env and gag proteins in a near-native conformation (Fang, et al. J. Infect. Dis. 180 (4): 1122-32 (1999)). In addition, intracellular processing of the HIV-1 proteins via the MHC class I pathway facilitates stimulation of cytotoxic T-lymphocytes. Part of the rationale for use of Gag from a subtype B in Thailand is that portions of the gag gene are conserved among virus subtypes. Therefore, gag-specific CTL elicited by vCP1521 may cross-react with CTL epitopes on non-subtype B primary viruses. Data from an AVEG-sponsored prime-boost trial (vCP205 alone or boosted with Chiron SF2 gp120/MF59) showed that CD8⁺ CTL from some vaccine recipients recognized target cells infected with non-subtype B viruses, including subtype E (Ferrari, et al. Proc. Natl. Acad. Sci. USA, 94:1396-401 (1997)).

In view of this data, several attempts have been made to provide protection using a prime-boost immunization format (McNeil, et al. Science. 303:961 (2004)). For example, ALVAC-HIV and AIDSVAX® B/E (VaxGen) have been used as the prime and boost compositions, respectively, and shown to induce neutralizing antibodies (Karnasuta, et al. Vaccine, 23: 2522-2529 (2005). In one safety trial, neutralizing antibodies were observed in 84% to 100% of subjects, cytotoxic lymphocytes (CTL) were observed in 16-25% of subjects, and lympho-proliferation was observed in 55-93% of subjects. In another safety trial, neutralizing antibodies were observed in 31% to 71% of subjects, cytotoxic lymphocytes (CTL) were observed in about 25% of subjects, and lympho-proliferation was observed in 58-71% of subjects. However, protection against infection by HIV was not shown in a large trial using the AIDSVAX component, leading some to question the value of such a combination vaccine (Burton, et al. Science. 303: 316 (2004); Letters to the Editor. Science. 305:177-180 (2004)).

Of the very few monoclonal antibodies with broadly neutralizing activity against HIV, three mAbs (2F5, 4E10 and Z13e1) recognize epitopes at the highly conserved membrane-proximal external region (MPER) of gp41. Exactly how these antibodies neutralize virus and to what Env structure they react to remains unclear. However, it appears that while these mAbs map to linear MPER sequences, a higher order structure contributes to antibody recognition. It has been described that 2F5 and 4E10 mAbs can bind to both native and fusion activated gp41 structures and much of the existing evidence suggests that these mAbs can neutralize the virus at different stages of infection. In addition, it has been described that lipid interactions with the long heavy chain CDR3 loop are important for increased recognition by these mAbs.

Other possible targets within gp41 to elicit a bNt response are the heptad repeat (HR) regions, in particular the highly conserved N-terminal HR(N-HR). The current model of Env-mediated HIV-1 infection suggests that binding of gp120 to CD4 and a co-receptor triggers a conformational change that dissociates gp120 from gp41. As a result, the fusion peptide (FP) at the extremity of gp41 is exposed and penetrates into the host membrane. This is followed by large conformational rearrangements within gp41 during which this protein adopts an energetically more favorable conformation, also known as a 6-helix bundle (6HB), consisting in an anti-parallel coiled-coil arrangement of three helices C-terminal HR region (C-HR) and a central trimer N-HR helices. This arrangement makes it possible for the viral membrane to fuse with the plasma membrane. In this model, the pre-fusogenic conformations of gp41 are characterized by the fact that the N-HR trimer and the three C-HR helices are exposed to solvents. This has been inferred from the observation that different molecules that bind to either the N-HR or C-HR regions, such as the T20 and other peptides, small compounds or the D5 mAb, are able to block gp41 re-arrangement and consequently inhibit HIV-cell fusion. Many recombinant constructs encompassing the gp41 ectodomain are very insoluble at neutral pH. Solubility is an important feature of potential immunological compositions and/or vaccines. Provided herein are several solutions to these problems, including derivatives of gp41 that are soluble and shown to produce anti-gp41 immune responses.

### BRIEF DESCRIPTION OF THE DRAWINGS

FIG. 1. gp41 polypeptide sequences.

FIG. 2. Comparison of amino acid sequence of FP-UGR7-MPR-A-2 and gp41.

FIG. 3. 6-helix-bundle conformation of gp41 polypeptides

FIG. 4. Thermal stability of gp41 polypeptides.

FIG. 5. A. Liposomes prepared in PBS/Tween 20. B. Liposomes prepared in PBS/β-OG.

FIG. 6. FP-UGR7-MPR-A-2 containing liposomes prepared in PB-Saccharose/Tween 20 at 55° C.

FIG. 7. SDS-PAGE of liposomal FP-UGR7-MPR-A-2 experiment 1.

### SUMMARY OF THE DISCLOSURE

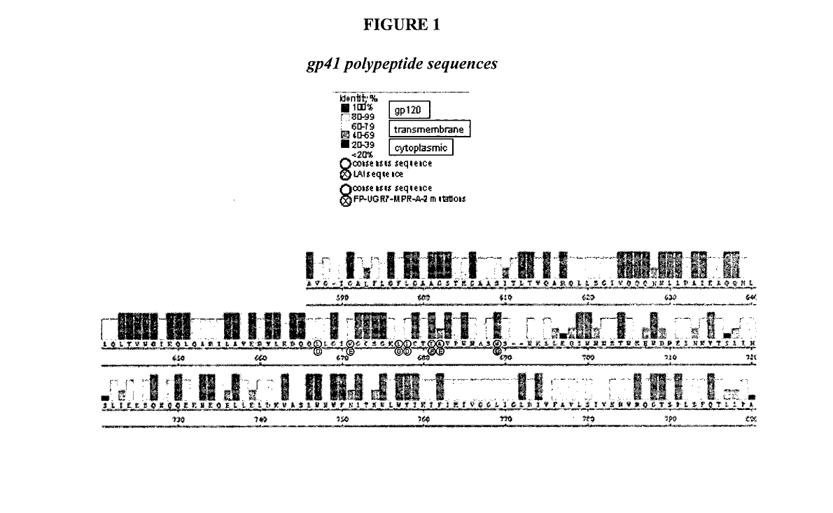

The reagents and methodologies described herein may be used to immunize a human being against human immunodeficiency virus. In some embodiments, a composition comprising a polypeptide and/or nucleic acid encoding the same is provided. In certain embodiments, the polypeptide may be a gp41 polypeptide modified to exhibit at least one characteristic relative to a wild-type gp41 polypeptide, the at least one characteristic being selected from the group consisting of reduced hydrophobicity, increased solubility at physiological pH, increased net charge, and decreased propensity to form a post-fusion conformation. The gp41 polypeptide in which the modifications may be made may be, for example, any of the gp41 polypeptides illustrated in FIG. 1 (e.g., SEQ ID NO.: 1). For example, the gp41 polypeptide may contain at least one amino acid substitution at, for example, leucine 81 (L81), tryptophan 85 (W85), threonine 95 (T95), alanine 96 (A96), leucine 91 (L91), isoleucine 92 (I92), tryptophan 103 (W103), and/or equivalents thereof. In certain embodiments, the at least one amino acid substitution may be selected from the group consisting of leucine 81 (L81), tryptophan 85 (W85), threonine 95 (T95), alanine 96 (A96), and/or equivalents thereof. In some embodiments, the amino acid substitution may be selected from the group consisting of leucine 91 (L91), isoleucine 92 (I92), tryptophan 103 (W103), and/or equivalents thereof. In some embodiments, a first at least one amino acid substitution may be at one or more of leucine 81 (L81), tryptophan 85 (W85), threonine 95 (T95), alanine 96 (A96), leucine 91 (L91), isoleucine 92 (I92), tryptophan 103 (W103), and/or equivalents thereof, while a second at least one substitution may be at, for example, leucine 91 (L91), isoleucine 92 (I92), tryptophan 103 (W103), and/or equivalents thereof. Exemplary substitutions may include, for example, L81 or equivalent thereof by aspartic acid (D) (L81D), W85 or equivalent thereof by glutamic acid (E) (W85E), L91 or equivalent thereof by glycine (G) (L91G), 192 or equivalent thereof by aspartic acid (D) (I92D), T95 or equivalent thereof by proline (P) (T95P), A96 or equivalent thereof by glutamic acid (E) (A96E), and/or W103 or equivalent thereof is by aspartic acid (D) (W103D). Any such isolated polypeptides may further comprise a deletion of the gp41 polar region (e.g. AGSTMGARSMTLTVQA (SEQ ID NO.: 3)). In certain embodiments, the polypeptide is SEQ ID NO.: 1 (e.g., AVGIGALFLGFLGARQLLSGIVQQQNNLLRAIEAQQHLLQLTVWGIKQLQARILAVERYLKDQQ DLGIEGCSGKGDCTPEVPWNASDSNKSLEQIWNNMTWMEWDREINNYTSLIHSLIEESQNQQEK NEQELLELDKWASLWNWFNITNWLW (substitutions underlined)). In some embodiments, the polypeptide may include the N-terminal amino acid sequence MHKVHGSGSGS (SEQ ID NO.: 2), which may assist with expression in recombinant systems (e.g., E. coli). In certain embodiments, the gp41 polypeptide may be prepared and/or utilized in trimeric form. In some embodiments, the polypeptide does not include the amino acid sequence AGSTMGARSMTLTVQA (SEQ ID NO.: 3). Any of these polypeptides may be termed FP-A (e.g. FP-UGR7_MPR-A-2, SEQ ID No. 1).

Some embodiments comprise nucleic acid sequences as well as expression vectors and/or host cells containing the same, and methods for expressing and producing the polypeptides using such nucleic acids, expression vectors, and/or host cells. Compositions comprising such gp41 polypeptides and/or nucleic acids encoding the same are also provided. In some embodiments, composition may comprise one or more adjuvants (e.g., monophosphoryl lipid A (MPLA)). In some embodiments, the compositions may be in the form of a liposome. Exemplary liposomes may comprise di-myristoyl-phosphatidylcholine (DMPC), cholesterol, and di-myristoyl-phosphatidylglycerol (DMPG). In certain embodiments, the molar ratio of DMPC to cholesterol to DMPG in the composition is about 9:7:1. Also provided are methods for producing such liposomes. In certain embodiments, the liposomes are produced by combining a lipid with the polypeptide in the presence of octyl-β-D-glucopyranoside (β-OG), Tween 20 and/or other suitable detergents, which may be necessary to solubilize and stabilize the hydrophobic membrane proteins. In some embodiments, the liposomes within a composition are of substantially similar sizes (e.g., an average diameter of approximately 70 to 130 nm).

Methods for producing an immune response against HIV using the gp41 polypeptides, nucleic acids, expression vectors, host cells, compositions, and/or liposomes are also provided. In some embodiments, the method may use an immunogenic composition to produce an immune response in a host to which the composition is administered. In others, the methods may use a vaccine composition to provide a protective and/or therapeutic immune response in a host to which the composition is administered.

Additional embodiments of the disclosure are described below. The embodiments described are to be considered limiting of this disclosure or the subject matter claimed herein.

### DETAILED DESCRIPTION

This disclosure provides compositions and methodologies useful for treating and/or preventing conditions relating to an infectious agent(s) such as a virus by stimulating an immune response against such an agent. In general, the immune response results from expression of an immunogen derived from or related to such an agent following administration of a nucleic acid vector encoding the immunogen, for example. In certain embodiments, multiple immunogens (which may be the same or different) are utilized. In other embodiments, variants and/or derivatives (i.e., by substitution, deletion or addition of amino acids or nucleotides encoding the same) of an immunogen or immunogens (which may be the same or different) may be utilized.

An immunogen may be a moiety (e.g., polypeptide, peptide or nucleic acid) that induces or enhances the immune response of a host to whom or to which the immunogen is administered. An immune response may be induced or enhanced by either increasing or decreasing the frequency, amount, or half-life of a particular immune modulator (e.g, the expression of a cytokine, chemokine, co-stimulatory molecule). This may be directly observed within a host cell containing a polynucleotide of interest (e.g., following infection by a recombinant virus) or within a nearby cell or tissue (e.g., indirectly). The immune response is typically directed against a target antigen. For example, an immune response may result from expression of an immunogen in a host following administration of a nucleic acid vector encoding the immunogen to the host. The immune response may result in one or more of an effect (e.g., maturation, proliferation, direct- or cross-presentation of antigen, gene expression profile) on cells of either the innate or adaptive immune system. For example, the immune response may involve, effect, or be detected in innate immune cells such as, for example, dendritic cells, monocytes, macrophages, natural killer cells, and/or granulocytes (e.g., neutrophils, basophils or eosinophils). The immune response may also involve, effect, or be detected in adaptive immune cells including, for example, lymphocytes (e.g., T cells and/or B cells). The immune response may be observed by detecting such involvement or effects including, for example, the presence, absence, or altered (e.g., increased or decreased) expression or activity of one or more immunomodulators such as a hormone, cytokine, interleukin (e.g., any of IL-1 through IL-35), interferon (e.g., any of IFN-I (IFN-α, IFN-β, IFN-ε, IFN-κ, IFN-τ, IFN-ζ, IFN-ω), IFN-II (e.g., IFN-γ), IFN-III (IFN-λ1, IFN-λ2, IFN-λ3)), chemokine (e.g., any CC cytokine (e.g., any of CCL1 through CCL28), any CXC chemokine (e.g., any of CXCL1 through CXCL24), Mip1a), any C chemokine (e.g., XCL1, XCL2), any CX3C chemokine (e.g., CX3CL1)), tumor necrosis factor (e.g., TNF-α, TNF-β)), negative regulators (e.g., PD-1, IL-T) and/or any of the cellular components (e.g., kinases, lipases, nucleases, transcription-related factors (e.g., IRF-1, IRF-7, STAT-5, NFKB, STAT3, STAT1, IRF-10), and/or cell surface markers suppressed or induced by such immunomodulators) involved in the expression of such immunomodulators. The presence, absence or altered expression may be detected within cells of interest or near those cells (e.g., within a cell culture supernatant, nearby cell or tissue in vitro or in vivo, and/or in blood or plasma). Administration of the immunogen may induce (e.g., stimulate a de novo or previously undetected response), or enhance or suppress an existing response against the immunogen by, for example, causing an increased antibody response (e.g., amount of antibody, increased affinity/avidity) or an increased cellular response (e.g., increased number of activated T cells, increased affinity/avidity of T cell receptors, cytoxicity including but not limited to antibody-dependent cellular cytotoxicity (ADCC), proliferation). In the case of HIV infections, no clear correlates of immunity have been associated with protection (especially protective immunity), but any of the measures described herein may be helpful in determining the usefulness of the compositions and methods described herein. Some immune responses may, in the case of a viral immunogen, lead to decreased viral load in, or lead to elimination of the virus from a host. In certain embodiments, the immune response may be protective (e.g., as may be provided by a vaccine), meaning that the immune response may be capable of preventing initiation or continued infection of or growth within a host and/or by eliminating an agent (e.g., a causative agent, such as HIV) from the host. In some instances, elimination of an agent from the host may mean that the vaccine is therapeutic. In some embodiments, a composition comprising an immunogen may be administered to a population of hosts (e.g., human beings) and determined to provide protective immunity to only a portion of that population. The composition may therefore be considered to protect a portion of that population (e.g., about 1/10, ¼, ⅓, ½, or ¾ of the population). The proportion of the population that is protected may be calculated and thereby provide the efficacy of the composition in that population (e.g., about 10%, 25%, 33%, 50%, or 75% efficacy).

In some embodiments, a method for immunizing and/or protectively immunizing (e.g., vaccinating) a human being against human immunodeficiency virus (HIV) by administering to the human being at least one dose of a composition comprising at least one gp41 polypeptide and/or at least one nucleic acid encoding the same is provided. Variations and derivatives of gp41 polypeptides may also be suitable, as are described herein and could be determined by one of skill in the art. In some embodiments, multiple compositions comprising the at least one gp41 polypeptide and/or at least one nucleic acids encoding the same may be administered, either together (e.g., at essentially the same time (e.g., simultaneously) to the same or different sites of a host) or separately (e.g., either in time or site of administration in the host).

In some embodiments, a composition comprising a gp41 polypeptide and/or nucleic acid encoding the same is provided. Such compositions may be used to induce and/or enhance an immune response against HIV. In certain embodiments, the polypeptide may be a gp41 polypeptide modified to exhibit at least one characteristic different from a wild-type gp41 polypeptide. The at least one characteristic may be any of, for example, reduced hydrophobicity, increased solubility at physiological pH, increased net charge, and decreased propensity to form a post-fusion conformation. The gp41 polypeptide in which the modifications are made may be, for example, any of the gp41 polypeptides illustrated in FIG. 1 (e.g., SEQ ID NO.: 1).

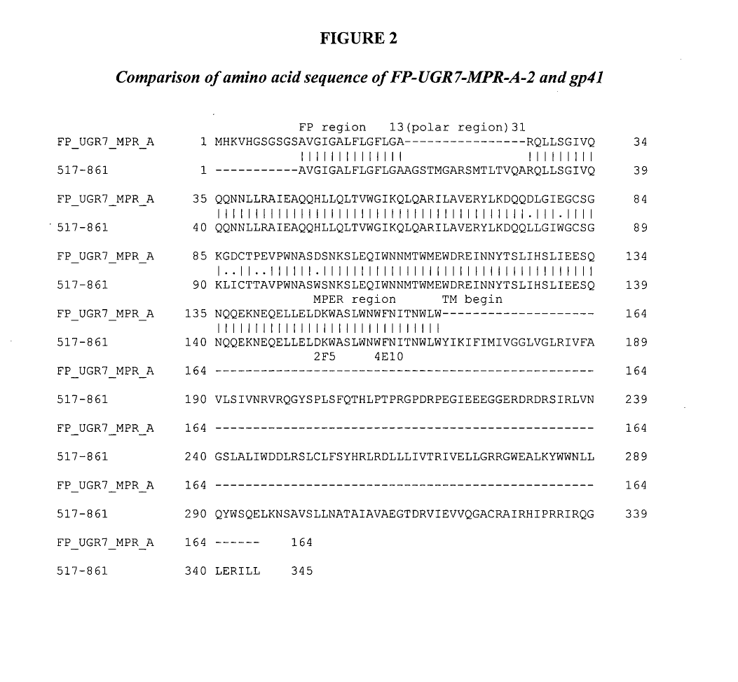

For example, the gp41 polypeptide may contain at least one amino acid substitution at, for example, leucine 81 (L81), tryptophan 85 (W85), threonine 95 (T95), alanine 96 (A96), leucine 91 (L91), isoleucine 92 (I92), tryptophan 103 (W103), and/or equivalents thereof. In certain embodiments, the at least one amino acid substitution may be at one or more of leucine 81 (L81), tryptophan 85 (W85), threonine 95 (T95), alanine 96 (A96), and/or equivalents thereof. In some embodiments, the amino acid substitution may be at one or more of leucine 91 (L91), isoleucine 92 (I92), tryptophan 103 (W103), and/or equivalents thereof. In some embodiments, a first amino acid substitution may be at one or more of leucine 81 (L81), tryptophan 85 (W85), threonine 95 (T95), alanine 96 (A96), leucine 91 (L91), isoleucine 92 (I92), tryptophan 103 (W103), and/or equivalents thereof, and a second substitution may be at one or more of, for example, leucine 91 (L91), isoleucine 92 (I92), tryptophan 103 (W103), and/or equivalents thereof. Exemplary substitutions may include, for example, L81 or equivalent thereof by aspartic acid (D) (L81D), W85 or equivalent thereof by glutamic acid (E) (W85E), L91 or equivalent thereof by glycine (G) (L91G), 192 or equivalent thereof by aspartic acid (D) (I92D), T95 or equivalent thereof by proline (P) (T95P), A96 or equivalent thereof by glutamic acid (E) (A96E), and/or W103 or equivalent thereof by aspartic acid (D) (W103D). Any such isolated gp41 polypeptides may further comprise a deletion of the gp41 polar region (e.g. AGSTMGARSMTLTVQA (SEQ ID NO.: 3); FIG. 2). In certain embodiments, the gp41 polypeptide is:

(substitutions to gp41 underlined;
SEQ ID NO.: 1; FIG. 2)
AVGIGALFLGFLGARQLLSGIVQQQNNLLRAIEAQQHLLQLTVWGIKQLQ
ARILAVERYLKDQQDLGIEGCSGKGDCTPEVPWNASDSNKSLEQIWNNMT
WMEWDREINNYTSLIHSLIEESQNQQEKNEQELLELDKWASLWNWFNITN
WLW.

In some embodiments, the gp41 polypeptide may include the N-terminal amino acid sequence MHKVHGSGSGS (SEQ ID NO.:2), which may assist with expression in recombinant systems (e.g., E. coli). In certain embodiments, the inclusion of the N-terminal amino acid sequence MHKVHGSGSGS (SEQ ID NO.: 2) significantly improves the level of expression of the polypeptide to which it is attached (e.g., SEQ ID NO.: 1). Any suitable host cell may be used to express the polypeptides described herein. For example, a suitable prokaryotic host cell may include those containing, for example, the DE3 prophage (e.g., BLR(DE3) (available from Novagen, reference: 69053), BL21(DE3), C41(DE3), C43(DE3)), and/or others (e.g., E. coli, AB1899, MM294, DH5α, JM109, H. halobium, K12, B834, BL21, Tuner, Origami, NovaBlue, cells described in U.S. Pat. Nos. 4,952,512; 4,929,553; 4,713,339; 4,711,848; and/or 4,704,362), and/or derivatives thereof. Other potential host cells include eukaryotic cells such as, for example, mammalian, yeast, fungal, and/or insect cells (e.g., as in U.S. Pat. Nos. 4,546,082; 4,599,311; 5,648,254). One skilled in the art will appreciate that any suitable expression plasmid and/or host cell may be used to express the polypeptides described herein. In certain embodiments, the gp41 polypeptide may be prepared and/or utilized in trimeric form. Some embodiments comprise nucleic acid sequences as well as expression vectors and/or host cells containing the same, and methods for expressing and producing the polypeptides using such nucleic acids, expression vectors, and/or host cells.

Compositions comprising such gp41 polypeptides and/or nucleic acids encoding the same are also provided. Preferably, the compositions comprising liposomes contain the polypeptide form (e.g., SEQ ID NO.: 1) of the immunogen (e.g., optionally also with an adjuvant). In some embodiments, composition may further comprise one or more adjuvants (e.g., monophosphoryl lipid A (MPLA)). In some embodiments, the compositions may be in the form of a liposome. The liposomes typically comprise phospholipids, either as a homogenous preparation (e.g., a single type of phospholipid) or a mixture of different phospholipids. For instance, phospholipids with different chain lengths (e.g., one or more of C14, C16, C18, C20, or natural phospholipids with mixed chain lengths) may be used. Mixtures of cholesterol(s) and lipid(s) at various ratios may also be used. In some embodiments, a phosphoplipid providing a negative surface charge to the liposome may be used (e.g., DMPG, DMPA, DOTAP, DOTMA). Exemplary liposomes may comprise di-myristoyl-phosphatidylcholine (DMPC), cholesterol, and/or di-myristoyl-phosphatidylglycerol (DMPG). Any suitable molar ratio of DMPC to cholesterol to DMPG may be used in the composition including, for example, about 5:3:1, 6:4:1, 7:5:1, 8:6:1, 9:7:1, 10:8:1, and the like. In certain embodiments, the molar ratio of DMPC to cholesterol to DMPG in the composition is about 9:7:1 (e.g., as in the Examples). The liposomes may also comprise a detergent (e.g., Tween-20). In certain embodiments, the liposomes are produced by combining a lipid with the polypeptide in the presence of Tween-20 and isolating the liposome. In some embodiments, the liposomes within a composition are of substantially similar sizes (e.g., an average diameter (e.g., z-average mean) of approximately any of 70 to 130, 70-80, 80-90, 90-100, 100-110, 110-120, and 120-130 nm). The liposomes also typically exhibit a suitable polydispersity index of, for example, approximately any of 0.1, 0.15, 0.20, 0.25, 0.30, 0.35 or 0.40. In some embodiments, the z-average mean is approximately 80 to 130 nm with a polydispersity index of about 0.25. These measurements may be made using any suitable method and/or equipment such as, for example, dynamic laser light scattering (e.g, using a Malvern Nano ZS which typic equipped with a 4 mW Helium/Neon laser at 633 nm wavelength and measures the liposome samples with the non-invasive backscatter technology at a detection angle of 173°). Typically, measurements are made at approximately 25° C. Other formulations may also suffice. In preferred embodiments, the liposomes are at approximately homogenous.

Methods for producing such liposomes are also provided. The liposomes may be prepared using methods described in, for example, U.S. Pat. No. 6,843,942 and/or those described herein (e.g., the Examples). In brief, the method may comprise an ethanol injection technique with a detergent dilution method. The ethanolic lipid solution may be injected into a micellar protein solution, accompanied by dilution with an appropriate buffer to reduce the detergent concentration. Precipitation of the lipid components in the aqueous phase after injection builds bilayer planar fragments which form lipidic vesicles in the next step. The detergent stabilized hydrophobic polypeptides are forced into the lipidic membranes due to reduction of the detergent concentration by dilution. Once this proteoliposomes are formed, the residual detergent, which intercalates within the lipid membranes, may be removed by dialysis or diafiltration. Variations of these methods, or other suitable methods, may also be utilized as would be understood by the skilled artisan.

For example, a lipid intermediate solution (e.g., intermediate liposome suspension) comprising DMPC, cholesterol and DMPG in a molar ratio of approximately 9:1:7 may be prepared using in 96% ethanol (Merck) to a final ethanol concentration in the aqueous phase of between 7.5 and 10% at an appropriate temperature (e.g., 55° C. independent of the temperature of the aqueous phase in order to obtain lipid solubilization). A suitable intermediate liposome suspension may comprise, for example, a lipid concentration of approximately 5 μmol/ml (e.g., 504.8 μmol dissolved in 7.5 ml ethanol by stirring). The intermediate liposome suspension may also be prepared by additionally mixing the initial suspension with (or preparing it simultaneously with) another buffer (e.g., PBS) comprising a detergent (e.g., β-octylglucoside (β-OG) or Tween-20; see, e.g., Table 5). Detergents may be used at any appropriate concentration such as, for example, about any of, for example, 0.05% to 2.0%, including but not limited to about any of 0.05%, 0.1%, 0.25%, 0.5%, 0.75%, 1.0%, 1.25%, 1.5%, 1.75%, or 2.0%. The process may also include the simultaneous dilution with the same or a different buffer (e.g., PBS). A polypeptide (e.g., the gp41 polypeptide FP-UGR7-MPR-A-2 (SEQ ID NO.: 1)) in, for example, a buffer comprising a detergent (e.g., 50 mM phosphate buffer, pH 7.5, containing 0.014 to 0.0015% Tween 20 (e.g., 0.01464%)) may also be prepared. The polypeptide solution may then be diluted using another buffer (e.g., PB-saccharose buffer (Na₂HPO₄.2H₂O (1.44 g/L), KCl (0.2 g/L), KH₂PO₄ (0.2 g/L) and saccharose (92.42 g/L)) to an appropriate concentration of polypeptide (e.g., 0.25 to 0.30 mg/ml). Other sugars, such as, for instance, trehalose and/or glucose may also be utilized in such a buffer with or without saccharose. The sugars may be used at any appropriate concentration (e.g., about any of 50, 100, or 150 g/L). In some embodiments, a mixture of a liposome intermediate solution and a polypeptide solution may be prepared by crossflow injection (e.g., injection module diameter of approximately 250 μm, 7.5% ethanol concentration in the intermediate liposome suspension, a volume ratio of injection to dilution buffer of 1:4 (e.g., 20 ml/80 ml), and a temperature of 55° C. (ethanol solution and aqueous phases)). Thus, this disclosure provides methods for producing an immunogenic liposome by combining an ethanolic lipid solution, a micellar protein solution comprising a polypeptide (e.g., FP-UGR7-MPR-A-2 (SEQ ID NO.: 1)) and a detergent (e.g., (β-OG) or Tween-20), and a buffer (e.g., PBS, PB-saccharose); precipitating the lipid components in the aqueous phase; and, removing residual detergent. As described herein, one or more adjuvants (e.g., MPLA) may also be introduced at an appropriate concentration (e.g., 0.1 to 1 mg/mL, such as, for example, any of about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mg/mL). In certain embodiments, adjuvant (e.g., MPLA) may be solubilised together with the lipid components in the ethanol fraction. Adjuvant (e.g., MPLA) may also be added in the micellar protein solution by co-solubilizing the MPLA together with the proteins. Typically, such processes provide liposome suspensions comprising liposomes of a suitable average diameter (e.g., about 70-130 nm such as 80-90 nm). The liposomes may then be further processed by, for example, filtration. The incorporation of polypeptide into the liposome may be measured at various steps by any suitable detection technique (e.g., SDS-PAGE, western blot of liposomal and filtrate samples). Variations of these techniques may also be suitable, as would be understood by one of skill in the art.

As described above, in some embodiments, multiple compositions (e.g., at least a first composition comprising a polypeptide and/or nucleic acid, and at least a second composition comprising a polypeptide and/or nucleic acid) may be administered to the host to produce an immune response. For instance, a first composition comprising a gp41 polypeptide and/or nucleic acid encoding the same may be administered once or repeatedly prior to or after at least one administration of the second composition (e.g., also comprising the gp41 polypeptide or other immunogen), where the time between administrations is of sufficient length to allow for the development of an immune response within the host. The immune response may or may not be detectable at that point. In certain embodiments, administration of either or both the first and second compositions is via a route selected from the group consisting of mucosal, intradermal, intramuscular, subcutaneous, via skin scarification, intranodal, or intratumoral. The dose of the compositions may vary, but in some embodiments, such as where a viral vector is utilized. Suitable viral vectors may include, for example, poxyiral vectors such as vaccinia, NYVAC, Modified Virus Ankara (MVA), avipox, canarypox, ALVAC, ALVAC(2), fowlpox, or TROVAC. The viral vector may be used to express a polypeptide described herein (e.g., SEQ ID NO.:1) in a cell.

The immunogens (e.g., modified or unmodified (e.g., to be modified) gp41 or other immunogens) may be selected from any HIV isolate (e.g., any primary or cultured HIV-1, HIV-2, and/or HIV-3 isolate, strain, or Glade). As is well-known in the art, HIV isolates are now classified into discrete genetic subtypes. HIV-1 is known to comprise at least ten subtypes (A1, A2, A3, A4, B, C, D, E, F1, F2, G, H, J and K) (Taylor et al, NEJM, 359(18):1965-1966 (2008)). HIV-2 is known to include at least five subtypes (A, B, C, D, and E). Subtype B has been associated with the HIV epidemic in homosexual men and intravenous drug users worldwide. Most HIV-1 immunogens, laboratory adapted isolates, reagents and mapped epitopes belong to subtype B. In sub-Saharan Africa, India, and China, areas where the incidence of new HIV infections is high, HIV-1 subtype B accounts for only a small minority of infections, and subtype HIV-1 C appears to be the most common infecting subtype. Thus, in certain embodiments, it may be preferable to select immunogens from particular subtypes (e.g., HIV-1 subtypes B and/or C). It may be desirable to include immunogens from multiple HIV subtypes (e.g., HIV-1 subtypes B and C, HIV-2 subtypes A and B, or a combination of HIV-1, HIV-2, and/or HIV-3 subtypes) in a single immunological composition along with the immunogens described here such as, for example, SEQ ID NO.: 1 (e.g., in a liposome). Suitable HIV immunogens include HIV envelope (env; e.g., NCBI Ref. Seq. NP_{—}057856), gag (e.g., p6, p7, p17, p24, GenBank AAD39400.1), the protease encoded by pol (e.g., UniProt P03366), nef (e.g., GenBank CAA41585.1; Shugars, et al. J. Virol. August 1993, pp. 4639-4650 (1993)), as well as variants, derivatives, and fusion proteins thereof, as described by, for example, Gomez et al. Vaccine, Vol. 25, pp. 1969-1992 (2007). Immunogens may be combined as desired (e.g., different immunogens, or the same immunogen derived from different strains). For instance, a single composition may comprise multiple types of modified gp41 polypeptides derived from different HIV strains. Where multiple HIV immunogens are used, the at least one additional HIV immunogen may be, for example, gag, pol, nef, a variant thereof, and a derivative thereof. Thus, in some embodiments, the first or second composition additionally contain at least one additional HIV immunogen selected from the group consisting of gag, the protease component encoded by pol, nef, a variant thereof, and a derivative thereof.

The modified gp41 polypeptides described herein may be derived from any HIV virus. For example, the modified gp41 polypeptides may be derived from any HIV-1, HIV-2, and/or HIV-3. The HIV-1 may be, for example, HIV-1 subtype A1, HIV-1 subtype A2, HIV-1 subtype A3, HIV-1 subtype A4, HIV-1 subtype B, HIV-1 subtype C, HIV-1 subtype D, HIV-1 subtype E, HIV-1 subtype F1, HIV-1 subtype F2, HIV-1 subtype G, HIV-1 subtype H, HIV-1 subtype J and HIV-1 subtype K. The HIV-2 may be, for example, HIV-2 subtype A, HIV-2 subtype B, HIV-2 subtype C, HIV-2 subtype D, and HIV-2 subtype E. The viral vector may encode, for example, at least one polypeptide selected from the group consisting of HIV gp120 MN 12-485, HIV gp120 A244 12-484, and HIV gp120 GNE8 12-477.

In preferred embodiments, vectors are used to transfer a nucleic acid sequence encoding a polypeptide to a cell. A vector is any molecule used to transfer a nucleic acid sequence to a host cell. In certain cases, an expression vector is utilized. An expression vector is a nucleic acid molecule that is suitable for transformation of a host cell and contains nucleic acid sequences that direct and/or control the expression of the transferred nucleic acid sequences. Expression includes, but is not limited to, processes such as transcription, translation, and splicing, if introns are present. Expression vectors typically comprise one or more flanking sequences operably linked to a heterologous nucleic acid sequence encoding a polypeptide. As used herein, the term operably linked refers to a linkage between polynucleotide elements in a functional relationship such as one in which a promoter or enhancer affects transcription of a coding sequence. Flanking sequences may be homologous (i.e., from the same species and/or strain as the host cell), heterologous (i.e., from a species other than the host cell species or strain), hybrid (i.e., a combination of flanking sequences from more than one source), or synthetic, for example.

In certain embodiments, it is preferred that the flanking sequence is a transcriptional regulatory region that drives high-level gene expression in the target cell. The transcriptional regulatory region may comprise, for example, a promoter, enhancer, silencer, repressor element, or combinations thereof. The transcriptional regulatory region may be either constitutive, tissue-specific, cell-type specific (i.e., the region is drives higher levels of transcription in a one type of tissue or cell as compared to another), or regulatable (i.e., responsive to interaction with a compound such as tetracycline). The source of a transcriptional regulatory region may be any prokaryotic or eukaryotic organism, any vertebrate or invertebrate organism, or any plant, provided that the flanking sequence functions in a cell by causing transcription of a nucleic acid within that cell. A wide variety of transcriptional regulatory regions may be utilized in practicing the embodiments described herein.

In some embodiments, derivatives of polypeptides, peptides, or polynucleotides incorporated into or expressed by the vectors described herein including, for example, fragments and/or variants thereof may be utilized. Derivatives may result from, for example, substitution, deletion, or addition of amino acids or nucleotides from or to the reference sequence (e.g., the parental sequence). A derivative of a polypeptide or protein, for example, typically refers to an amino acid sequence that is altered with respect to the referenced polypeptide or peptide. A derivative of a polypeptide typically retains at least one activity of the polypeptide. A derivative will typically share at least approximately 60%, 70%, 80%, 90%, 95%, or 99% identity to the reference sequence. With respect to polypeptides and peptides, the derivative may have “conservative” changes, wherein a substituted amino acid has similar structural or chemical properties. A derivative may also have “nonconservative” changes. Exemplary, suitable conservative amino acid substitutions may include, for example, those shown in Table 1:

TABLE 1

| Original | | Preferred |
|---|---|---|
| Residues | Exemplary Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, 1,4 Diamino-butyric Acid, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

Other amino acid substitutions may be considered non-conservative. Derivatives may also include amino acid or nucleotide deletions and/or additions/insertions, or some combination of these. Guidance in determining which amino acid residues or nucleotides may be substituted, inserted, or deleted without abolishing the desired activity of the derivative may be identified using any of the methods available to one of skill in the art.

Derivatives may also refer to a chemically modified polynucleotide or polypeptide. Chemical modifications of a polynucleotide may include, for example, replacement of hydrogen by an alkyl, acyl, hydroxyl, or amino group. A derivative polynucleotide may encode a polypeptide which retains at least one biological or immunological function of the natural molecule. A derivative polypeptide may be one modified by glycosylation, pegylation, biotinylation, or any similar process that retains at least one biological or immunological function of the polypeptide from which it was derived.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 10, at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured. Percent identity can be measured both globally or locally. Examples of alignment algorithms known in the art for global alignments are ones which attempt to align every residue in every sequence, such as the Needleman-Wunsch algorithm. Local alignment algorithmns are useful for dissimilar sequences that contain regions of similar sequence motifs within their larger sequence, such as the Smith-Waterman algorithm.

As mentioned above, this disclosure relates to compositions comprising recombinant vectors, the vectors per se, and methods of using the same. A “vector” is any moiety (e.g., a virus or plasmid) used to carry, introduce, or transfer a polynucleotide or interest to another moiety (e.g., a host cell). In certain cases, an expression vector is utilized. An expression vector is a nucleic acid molecule containing a polynucleotide of interest encoding a polypeptide, peptide, or polynucleotide and also containing other polynucleotides that direct and/or control the expression of the polynucleotide of interest. Expression includes, but is not limited to, processes such as transcription, translation, and/or splicing (e.g., where introns are present).

Viral vectors that may be used include, for example, retrovirus, adenovirus, adeno-associated virus (AAV), alphavirus, herpes virus, and poxvirus vectors, among others. Many such viral vectors are available in the art. The vectors described herein may be constructed using standard recombinant techniques widely available to one skilled in the art. Such techniques may be found in common molecular biology references such as Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), and PCR Protocols: A Guide to Methods and Applications (Innis, et al. 1990. Academic Press, San Diego, Calif.).

Suitable retroviral vectors may include derivatives of lentivirus as well as derivatives of murine or avian retroviruses. Examplary, suitable retroviral vectors may include, for example, Moloney murine leukemia virus (MoMuLV), Harvey murine sarcoma virus (HaMuSV), murine mammary tumor virus (MuMTV), SIV, BIV, HIV and Rous Sarcoma Virus (RSV). A number of retroviral vectors can incorporate multiple exogenous polynucleotides. As recombinant retroviruses are defective, they require assistance in order to produce infectious vector particles. This assistance can be provided by, for example, helper cell lines encoding retrovirus structural genes. Suitable helper cell lines include Ψ2, PA317 and PA12, among others. The vector virions produced using such cell lines may then be used to infect a tissue cell line, such as NIH 3T3 cells, to produce large quantities of chimeric retroviral virions. Retroviral vectors may be administered by traditional methods (i.e., injection) or by implantation of a “producer cell line” in proximity to the target cell population (Culver, K., et al., 1994, Hum. Gene Ther., 5 (3): 343-79; Culver, K., et al., Cold Spring Harb. Symp. Quant. Biol., 59: 685-90); Oldfield, E., 1993, Hum. Gene Ther., 4 (1): 39-69). The producer cell line is engineered to produce a viral vector and releases viral particles in the vicinity of the target cell. A portion of the released viral particles contact the target cells and infect those cells, thus delivering a nucleic acid encoding an immunogen to the target cell. Following infection of the target cell, expression of the polynucleotide of interest from the vector occurs.

Adenoviral vectors have proven especially useful for gene transfer into eukaryotic cells (Rosenfeld, M., et al., 1991, Science, 252 (5004): 431-4; Crystal, R., et al., 1994, Nat. Genet., 8 (1): 42-51), the study eukaryotic gene expression (Levrero, M., et al., 1991, Gene, 101 (2): 195-202), vaccine development (Graham, F. and Prevec, L., 1992, Biotechnology, 20: 363-90), and in animal models (Stratford-Perricaudet, L., et al., 1992, Bone Marrow Transplant., 9 (Suppl. 1): 151-2; Rich, et al., 1993, Hum. Gene Ther., 4 (4): 461-76). Experimental routes for administrating recombinant Ad to different tissues in vivo have included intratracheal instillation (Rosenfeld, M., et al., 1992, Cell, 68 (1): 143-55) injection into muscle (Quantin, B., et al., 1992, Proc. Natl. Acad. Sci. U.S.A., 89 (7): 2581-4), peripheral intravenous injection (Herz, J., and Gerard, R., 1993, Proc. Natl. Acad. Sci. U.S.A., 90 (7): 2812-6) and/or stereotactic inoculation to brain (Le Gal La Salle, G., et al., 1993, Science, 259 (5097): 988-90), among others.

Adeno-associated virus (AAV) demonstrates high-level infectivity, broad host range and specificity in integrating into the host cell genome (Hermonat, P., et al., 1984, Proc. Natl. Acad. Sci. U.S.A., 81 (20): 6466-70). And Herpes Simplex Virus type-1 (HSV-1) is yet another attractive vector system, especially for use in the nervous system because of its neurotropic property (Geller, A., et al., 1991, Trends Neurosci., 14 (10): 428-32; Glorioso, et al., 1995, Mol. Biotechnol., 4 (1): 87-99; Glorioso, et al., 1995, Annu. Rev. Microbiol., 49: 675-710).

Alphavirus may also be used to express the immunogen in a host. Suitable members of the Alphavirus genus include, among others, Sindbis virus, Semliki Forest virus (SFV), the Ross River virus and Venezuelan, Western and Eastern equine encephalitis viruses, among others. Expression systems utilizing alphavirus vectors are described in, for example, U.S. Pat. Nos. 5,091,309; 5,217,879; 5,739,026; 5,766,602; 5,843,723; 6,015,694; 6,156,558; 6,190,666; 6,242,259; and, 6,329,201; WO 92/10578; Xiong et al., Science, Vol 243, 1989, 1188-1191; Liliestrom, et al. Bio/Technology, 9: 1356-1361, 1991. Thus, the use of alphavirus as an expression system is well known by those of skill in the art.

Poxvirus is another useful expression vector (Smith, et al. 1983, Gene, 25 (1): 21-8; Moss, et al, 1992, Biotechnology, 20: 345-62; Moss, et al, 1992, Curr. Top. Microbiol. Immunol., 158: 25-38; Moss, et al. 1991. Science, 252: 1662-1667). The most often utilized poxyiral vectors include vaccinia and derivatives therefrom such as NYVAC and MVA, and members of the avipox genera such as fowlpox, canarypox, ALVAC, and ALVAC(2), among others.

An exemplary suitable vector is NYVAC (vP866) which was derived from the Copenhagen vaccine strain of vaccinia virus by deleting six nonessential regions of the genome encoding known or potential virulence factors (see, for example, U.S. Pat. Nos. 5,364,773 and 5,494,807). The deletion loci were also engineered as recipient loci for the insertion of foreign genes. The deleted regions are: thymidine kinase gene (TK; J2R); hemorrhagic region (u; B13R+B 14R); A type inclusion body region (ATI; A26L); hemagglutinin gene (HA; A56R); host range gene region (C7L-K1L); and, large subunit, ribonucleotide reductase (14L). NYVAC is a genetically engineered vaccinia virus strain that was generated by the specific deletion of eighteen open reading frames encoding gene products associated with virulence and host range.

NYVAC has been show to be useful for expressing TAs (see, for example, U.S. Pat. No. 6,265,189). NYVAC (vP866), vP994, vCP205, vCP1433, placZH6H4Lreverse, pMPC6H6K3E3 and pC3H₆FHVB were also deposited with the ATCC under the terms of the Budapest Treaty, accession numbers VR-2559, VR-2558, VR-2557, VR-2556, ATCC-97913, ATCC-97912, and ATCC-97914, respectively.

Another suitable virus is the Modified Vaccinia Ankara (MVA) virus which was generated by 516 serial passages on chicken embryo fibroblasts of the Ankara strain of vaccinia virus (CVA) (for review, see Mayr, A., et al. Infection 3, 6-14 (1975)). It was shown in a variety of animal models that the resulting MVA was significantly avirulent (Mayr, A. & Danner, K. (1978) Dev. Biol. Stand. 41: 225.34) and has been tested in clinical trials as a smallpox vaccine (Mayr et al., Zbl. Bakt. Hyg. I, Abt. Org. B 167, 375-390 (1987), Stickl et al., Dtsch. med. Wschr. 99, 2386-2392 (1974)). MVA has also been engineered for use as a viral vector for both recombinant gene expression studies and as a recombinant vaccine (Sutter, G. et al. (1994), Vaccine 12: 1032-40; Blanchard et al., 1998, J Gen Virol 79, 1159-1167; Carroll & Moss, 1997, Virology 238, 198-211; Altenberger, U.S. Pat. No. 5,185,146; Ambrosini et al., 1999, J Neurosci Res 55(5), 569). Modified virus Ankara (MVA) has been previously described in, for example, U.S. Pat. Nos. 5,185,146 and 6,440,422; Sutter, et al. (B. Dev. Biol. Stand. Base1, Karger 84:195-200 (1995)); Antoine, et al. (Virology 244: 365-396, 1998); Sutter et al. (Proc. Natl. Acad. Sci. USA 89: 10847-10851, 1992); Meyer et al. (J. Gen. Virol. 72: 1031-1038, 1991); Mahnel, ett al. (Berlin Munch. Tierarztl. Wochenschr. 107: 253-256, 1994); Mayr et al. (Zbl. Bakt. Hyg. I, Abt. Org. B 167: 375-390 (1987); and, Stickl et al. (Dtsch. med. Wschr. 99: 2386-2392 (1974)). An exemplary MVA is available from the ATCC under accession numbers VR-1508 and VR-1566.

ALVAC-based recombinant viruses (i.e., ALVAC-1 and ALVAC-2) are also suitable for use as described herien (see, for example, U.S. Pat. No. 5,756,103). ALVAC(2) is identical to ALVAC(1) except that ALVAC(2) genome comprises the vaccinia E3L and K3L genes under the control of vaccinia promoters (U.S. Pat. No. 6,130,066; Beattie et al., 1995a, 1995b, 1991; Chang et al., 1992; Davies et al., 1993). Both ALVAC(1) and ALVAC(2) have been demonstrated to be useful in expressing foreign DNA sequences, such as TAs (Tartaglia et al., 1993 a,b; U.S. Pat. No. 5,833,975). ALVAC was deposited under the terms of the Budapest Treaty with the American Type Culture Collection (ATCC), 10801 University Boulevard, Manassas, Va. 20110-2209, USA, ATCC accession number VR-2547. Vaccinia virus host range genes (e.g., C18L, C17L, C7L, K1L, E3L, B4R, B23R, and B24R) have also been shown to be expressible in canarypox (e.g., U.S. Pat. No. 7,473,536).

Another useful poxvirus vector is TROVAC. TROVAC refers to an attenuated fowlpox that was a plaque-cloned isolate derived from the FP-1 vaccine strain of fowlpoxvirus which is licensed for vaccination of 1 day old chicks. TROVAC was likewise deposited under the terms of the Budapest Treaty with the ATCC, accession number 2553.

“Non-viral” plasmid vectors may also be suitable for use. Plasmid DNA molecules comprising expression cassettes for expressing an immunogen may be used for “naked DNA” immunization. Preferred plasmid vectors are compatible with bacterial, insect, and/or mammalian host cells. Such vectors include, for example, PCR-II, pCR3, and pcDNA3.1 (Invitrogen, San Diego, Calif.), pBSII (Stratagene, La Jolla, Calif.), pET15 (Novagen, Madison, Wis.), pGEX (Pharmacia Biotech, Piscataway, N.J.), pEGFP-N2 (Clontech, Palo Alto, Calif.), pETL (BlueBacII, Invitrogen), pDSR-alpha (PCT pub. No. WO 90/14363) and pFastBacDual (Gibco-BRL, Grand Island, N.Y.) as well as Bluescript® plasmid derivatives (a high copy number COLE1-based phagemid, Stratagene Cloning Systems, La Jolla, Calif.), PCR cloning plasmids designed for cloning Taq-amplified PCR products (e.g., TOPO™ TA Cloning® cloning kit, PCR2.1® plasmid derivatives, Invitrogen, Carlsbad, Calif.).

Bacterial vectors may also be suitable for use. These vectors include, for example, Shigella, Salmonella (e.g., Darji, et al. Cell, 91: 765-775 (1997); Woo, et al. Vaccine, 19: 2945-2954 (2001)), Vibrio cholerae, Lactobacillus, Bacille calmette guérin (BCG), and Streptococcus (e.g., WO 88/6626, WO 90/0594, WO 91/13157, WO 92/1796, and WO 92/21376). Many other non-viral plasmid expression vectors and systems are known in the art and could be used as described herein.

Nucleic acid delivery or transformation techniques that may be used include DNA-ligand complexes, adenovirus-ligand-DNA complexes, direct injection of DNA, CaPO₄ precipitation, gene gun techniques, electroporation, and colloidal dispersion systems, among others. Colloidal dispersion systems include macromolecule complexes, nanocapsules, microspheres, beads, and lipid-based systems including oil-in-water emulsions, micelles, mixed micelles, and liposomes. The preferred colloidal system may be a liposome, which are artificial membrane vesicles useful as delivery vehicles in vitro and in vivo. RNA, DNA and intact virions can be encapsulated within the aqueous interior and be delivered to cells in a biologically active form (Fraley, R., et al. Trends Biochem. Sci., 6: 77 (1981)). The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from approximately 12 to 20 carbon atoms, particularly from 14-18 carbon atoms, and is saturated. Illustrative phospholipids include egg phosphatidylcholine, dipalmitoylphosphatidylcholine and distearoylphosphatidylcholine.

Strategies for improving the efficiency of nucleic acid-based immunization may also be used including, for example, the use of self-replicating viral replicons (Caley, et al. Vaccine, 17: 3124-2135 (1999); Dubensky, et al. Mol. Med. 6: 723-732 (2000); Leitner, et al. Cancer Res. 60: 51-55 (2000)), codon optimization (Liu, et al. Mol. Ther., 1: 497-500 (2000); Dubensky, supra; Huang, et al. J. Virol. 75: 4947-4951 (2001)), in vivo electroporation (Widera, et al. J. Immunol. 164: 4635-3640 (2000)), incorporation of CpG stimulatory motifs (Gurunathan, et al. Ann. Rev. Immunol. 18: 927-974 (2000); Leitner, supra), sequences for targeting of the endocytic or ubiquitin-processing pathways (Thomson, et al. J. Virol. 72: 2246-2252 (1998); Velders, et al. J. Immunol. 166: 5366-5373 (2001)), and/or prime-boost regimens (Gurunathan, supra; Sullivan, et al. Nature, 408: 605-609 (2000); Hanke, et al. Vaccine, 16: 439-445 (1998); Amara, et al. Science, 292: 69-74 (2001)). Other methods are known in the art, some of which are described below.

In other embodiments, it may be advantageous to combine or include within the compositions or recombinant vectors additional polypeptides, peptides or polynucleotides encoding one or more polypeptides or peptides that function as “co-stimulatory” component(s). Such co-stimulatory components may include, for example, cell surface proteins, cytokines or chemokines in a composition. The co-stimulatory component may be included in the composition as a polypeptide or peptide, or as a polynucleotide encoding the polypeptide or peptide, for example. Suitable co-stimulatory molecules include, for instance, polypeptides that bind members of the CD28 family (i.e., CD28, ICOS; Hutloff, et al. Nature 1999, 397: 263-265; Peach, et al. J Exp Med 1994, 180: 2049-2058) such as the CD28 binding polypeptides B7.1 (CD80; Schwartz, 1992; Chen et al, 1992; Ellis, et al. J. Immunol., 156(8): 2700-9) and B7.2 (CD86; Ellis, et al. J. Immunol., 156(8): 2700-9); polypeptides which bind members of the integrin family (i.e., LFA-1 (CD11a/CD18); Sedwick, et al. J Immunol 1999, 162: 1367-1375; Wülfing, et al. Science 1998, 282: 2266-2269; Lub, et al. Immunol Today 1995, 16: 479-483) including members of the ICAM family (i.e., ICAM-1, -2 or -3); polypeptides which bind CD2 family members (i.e., CD2, signalling lymphocyte activation molecule (CDw150 or “SLAM”; Aversa, et al. J Immunol 1997, 158: 4036-4044) such as CD58 (LFA-3; CD2 ligand; Davis, et al. Immunol Today 1996, 17: 177-187) or SLAM ligands (Sayos, et al. Nature 1998, 395: 462-469); polypeptides which bind heat stable antigen (HSA or CD24; Zhou, et al. Eur J Immunol 1997, 27: 2524-2528); polypeptides which bind to members of the TNF receptor (TNFR) family (i.e., 4-1BB (CD137; Vinay, et al. Semin Immunol 1998, 10: 481-489)), OX40 (CD134; Weinberg, et al. Semin Immunol 1998, 10: 471-480; Higgins, et al. J Immunol 1999, 162: 486-493), and CD27 (Lens, et al. Semin Immunol 1998, 10: 491-499)) such as 4-1BBL (4-1BB ligand; Vinay, et al. Semin Immunol 1998, 10: 481-48; DeBenedette, et al. J Immunol 1997, 158: 551-559), TNFR associated factor-1 (TRAF-1; 4-1BB ligand; Saoulli, et al. J Exp Med 1998, 187: 1849-1862, Arch, et al. Mol Cell Biol 1998, 18: 558-565), TRAF-2 (4-1BB and OX40 ligand; Saoulli, et al. J Exp Med 1998, 187: 1849-1862; Oshima, et al. Int Immunol 1998, 10: 517-526, Kawamata, et al. J Biol Chem 1998, 273: 5808-5814), TRAF-3 (4-1BB and OX40 ligand; Arch, et al. Mol Cell Biol 1998, 18: 558-565; Jang, et al. Biochem Biophys Res Commun 1998, 242: 613-620; Kawamata S, et al. J Biol Chem 1998, 273: 5808-5814), OX40L (OX40 ligand; Gramaglia, et al. J Immunol 1998, 161: 6510-6517), TRAF-5 (OX40 ligand; Arch, et al. Mol Cell Biol 1998, 18: 558-565; Kawamata, et al. J Biol Chem 1998, 273: 5808-5814), and CD70 (CD27 ligand; Couderc, et al. Cancer Gene Ther., 5(3): 163-75). CD154 (CD40 ligand or “CD40L”; Gurunathan, et al. J. Immunol., 1998, 161: 4563-4571; Sine, et al. Hum. Gene Ther., 2001, 12: 1091-1102) Other co-stimulatory molecules may also be suitable for use.

One or more cytokines may also be suitable co-stimulatory components or “adjuvants”, either as polypeptides or being encoded by nucleic acids contained within the compositions described herein (Parmiani, et al. Immunol Lett 2000 Sep. 15; 74(1): 41-4; Berzofsky, et al. Nature Immunol. 1: 209-219). Suitable cytokines include, for example, interleukin-2 (IL-2) (Rosenberg, et al. Nature Med. 4: 321-327 (1998)), IL-4, IL-7, IL-12 (reviewed by Pardoll, 1992; Harries, et al. J. Gene Med. 2000 July-August; 2(4):243-9; Rao, et al. J. Immunol. 156: 3357-3365 (1996)), IL-15 (Xin, et al. Vaccine, 17:858-866, 1999), IL-16 (Cruikshank, et al. J. Leuk Biol. 67(6): 757-66, 2000), IL-18 (J. Cancer Res. Clin. Oncol. 2001. 127(12): 718-726), GM-CSF (CSF (Disis, et al. Blood, 88: 202-210 (1996)), tumor necrosis factor-alpha (TNF-α), or interferon-gamma (INF-γ). Other cytokines may also be suitable for use.

Chemokines may also be utilized. For example, fusion proteins comprising CXCL10 (IP-10) and CCL7 (MCP-3) fused to a tumor self-antigen have been shown to induce anti-tumor immunity (Biragyn, et al. Nature Biotech. 1999, 17: 253-258). The chemokines CCL3 (MIP-1α) and CCL5 (RANTES) (Boyer, et al. Vaccine, 1999, 17 (Supp. 2): S53-S64) may also be of use. Other suitable chemokines are known in the art.

It is also known in the art that suppressive or negative regulatory immune mechanisms may be blocked, resulting in enhanced immune responses. For instance, treatment with anti-CTLA-4 (Shrikant, et al. Immunity, 1996, 14: 145-155; Sutmuller, et al. J. Exp. Med., 2001, 194: 823-832), anti-CD25 (Sutmuller, supra), anti-CD4 (Matsui, et al. J. Immunol., 1999, 163: 184-193), the fusion protein IL13Ra2-Fc (Terabe, et al. Nature Immunol., 2000, 1: 515-520), and combinations thereof (i.e., anti-CTLA-4 and anti-CD25, Sutmuller, supra) have been shown to upregulate anti-tumor immune responses and would be suitable.

An immunogen may also be administered in combination with one or more adjuvants to boost the immune response. Adjuvants may also be included to stimulate or enhance the immune response against the immunogen. Non-limiting examples of suitable adjuvants include those of the gel-type (i.e., aluminum hydroxide/phosphate (“alum adjuvants”), calcium phosphate), of microbial origin (muramyl dipeptide (MDP)), bacterial exotoxins (cholera toxin (CT), native cholera toxin subunit B (CTB), E. coli labile toxin (LT), pertussis toxin (PT), CpG oligonucleotides, BCG sequences, tetanus toxoid, monophosphoryl lipid A (MPLA) of, for example, E. coli, Salmonella minnesota, Salmonella typhimurium, or Shigella exseri, particulate adjuvants (biodegradable, polymer microspheres), immunostimulatory complexes (ISCOMs)), oil-emulsion and surfactant-based adjuvants (Freund's incomplete adjuvant (FIA), microfluidized emulsions (MF59, SAF), saponins (QS-21)), synthetic (muramyl peptide derivatives (murabutide, threony-MDP)), nonionic block copolymers (L121), polyphosphazene (PCCP), synthetic polynucleotides (poly A:U, poly I:C), thalidomide derivatives (CC-4407/ACTIMID)), RH3-ligand, or polylactide glycolide (PLGA) microspheres, among others. Fragments, homologs, derivatives, and fusions to any of these toxins are also suitable, provided that they retain adjuvant activity. Suitable mutants or variants of adjuvants are described, e.g., in WO 95/17211 (Arg-7-Lys CT mutant), WO 96/6627 (Arg-192-Gly LT mutant), and WO 95/34323 (Arg-9-Lys and Glu-129-Gly PT mutant). Additional LT mutants that can be used in the methods and compositions of the inventionas described herein may include, e.g., Ser-63-Lys, Ala-69-Gly, Glu-110-Asp, and Glu-112-Asp mutants. Other suitable adjuvants are also well-known in the art.

As an example, metallic salt adjuvants such alum adjuvants are well-known in the art as providing a safe excipient with adjuvant activity. The mechanism of action of these adjuvants are thought to include the formation of an antigen depot such that antigen may stay at the site of injection for up to 3 weeks after administration, and also the formation of antigen/metallic salt complexes which are more easily taken up by antigen presenting cells. In addition to aluminium, other metallic salts have been used to adsorb antigens, including salts of zinc, calcium, cerium, chromium, iron, and berilium. The hydroxide and phosphate salts of aluminium are the most common. Formulations or compositions containing aluminium salts, antigen, and an additional immunostimulant are known in the art. An example of an immunostimulant is 3-de-O-acylated monophosphoryl lipid A (3D-MPL).

Any of these components may be used alone or in combination with other agents. For instance, it has been shown that a combination of CD80, ICAM-1 and LFA-3 (“TRICOM”) may potentiate anti-cancer immune responses (Hodge, et al. Cancer Res. 59: 5800-5807 (1999). Other effective combinations include, for example, IL-12+GM-CSF (Ahlers, et al. J. Immunol., 158: 3947-3958 (1997); Iwasaki, et al. J. Immunol. 158: 4591-4601 (1997)), IL-12+GM-CSF+TNF-α (Ahlers, et al. Int. Immunol. 13: 897-908 (2001)), CD80+IL-12 (Fruend, et al. Int. J. Cancer, 85: 508-517 (2000); Rao, et al. supra), and CD86+GM-CSF+IL-12 (Iwasaki, supra). One of skill in the art would be aware of additional combinations useful in carrying out the embodiments described herein. In addition, the skilled artisan would be aware of additional reagents or methods that may be used to modulate such mechanisms. These reagents and methods, as well as others known by those of skill in the art, may also be utilized as described herein.

Other agents that may be utilized in conjunction with the compositions and methods provided herein include anti-HIV agents including, for example, protease inhibitor, an HIV entry inhibitor, a reverse transcriptase inhibitor, and/or or an anti-retroviral nucleoside analog. Suitable compounds include, for example, Agenerase (amprenavir), Combivir (Retrovir/Epivir), Crixivan (indinavir), Emtriva (emtricitabine), Epivir (3tc/lamivudine), Epzicom, Fortovase/Invirase (saquinavir), Fuzeon (enfuvirtide), Hivid (ddc/zalcitabine), Kaletra (lopinavir), Lexiva (Fosamprenavir), Norvir (ritonavir), Rescriptor (delavirdine), Retrovir/AZT (zidovudine), Reyatax (atazanavir, BMS-232632), Sustiva (efavirenz), Trizivir (abacavir/zidovudine/lamivudine), Truvada (Emtricitabine/Tenofovir DF), Videx (ddI/didanosine), Videx EC (ddI, didanosine), Viracept (nevirapine), Viread (tenofovir disoproxil fumarate), Zerit (d4T/stavudine), and Ziagen (abacavir). Other suitable agents are known to those of skill in the art. Such agents may either be used prior to, during, or after administration of the compositions and/or use of the methods described herein.

Administration of a composition to a host may be accomplished using any of a variety of techniques known to those of skill in the art. The composition(s) may be processed in accordance with conventional methods of pharmacy to produce medicinal agents for administration to patients, including humans and other mammals (i.e., a “pharmaceutical composition”). The pharmaceutical composition is preferably made in the form of a dosage unit containing a given amount of DNA, viral vector particles, polypeptide, peptide, or other drug candidate, for example. A suitable daily dose for a human or other mammal may vary widely depending on the condition of the patient and other factors, but, once again, can be determined using routine methods. The compositions are administered to a patient in a form and amount sufficient to elicit a therapeutic effect. Amounts effective for this use will depend on various factors, including for example, the particular composition of the vaccine regimen administered, the manner of administration, the stage and severity of the disease, the general state of health of the patient, and the judgment of the prescribing physician. The dosage regimen for immunizing a host or otherwise treating a disorder or a disease with a composition may be based on a variety of factors, including the type of disease, the age, weight, sex, medical condition of the patient, the severity of the condition, the route of administration, and the particular compound employed. Thus, the dosage regimen may vary widely, but can be determined routinely using standard methods.

In general, recombinant viruses may be administered in compositions in a dosage amount of about 10⁴ to about 10⁹ pfu per inoculation; often about 10⁴ pfu to about 10⁶ pfu, or as shown in the Examples, 10⁷ to 10³ pfu. Higher dosages such as about 10⁴ pfu to about 10¹⁰ pfu, e.g., about 10⁵ pfu to about 10⁹ pfu, or about 10⁶ pfu to about 10⁸ pfu, or about 10⁷ pfu can also be employed. Another measure commonly used is cell culture infective dose (CCID₅₀); suitable CCID₅₀ ranges for administration include about 10¹, about 10², about 10³, about 10⁴, about 10⁵, about 10⁶, about 10⁷, about 10⁸, about 10⁹, about 10¹⁰ CCID₅₀. Ordinarily, suitable dosage amounts of plasmid or naked DNA are about 1 μg to about 100 mg, about 1 mg, about 2 mg, but lower levels such as 0.1 to 1 mg or 1-10 μg may be employed. For polypeptide compositions, a suitable amount may be 1-1000 μg. Without limiting the possible sub-ranges within that dosage range, particular embodiments may employ 5, 10, 20, 50, 100, 150, 200, 250, 300, 350, 400, 450, 500, 550, 600, 650, 700, 750, 800, 850, 900, 950, and 1000 μg. A typical exemplary dosage of polypeptide may be, for example, about 50-250 μg, about 250-500 μg, 500-750 μg, or about 1000 μg of polypeptide. Low dose administration may typically utilize a dose of about 100 μg or less. High dose administration may typically utilize a dose of 300 μg or more. In referring to the amount of polypeptide in a dose, it is to be understood that the amount may refer to the amount of a single polypeptide or, where multiple polypeptides are administered, to the total amount of all polypeptides. “Dosage” may refer to that administered in a single or multiple doses, including the total of all doses administered. Actual dosages of such compositions can be readily determined by one of ordinary skill in the field of vaccine technology.

The pharmaceutical composition may be administered nasally (e.g., as may be used for EN41-FPA2), orally, vaginally, parenterally, by inhalation spray, rectally, intranodally, or topically in dosage unit formulations containing conventional pharmaceutically acceptable carriers, adjuvants, and vehicles. The term “pharmaceutically acceptable carrier” or “physiologically acceptable carrier” as used herein refers to one or more formulation materials suitable for accomplishing or enhancing the delivery of a nucleic acid, polypeptide, or peptide as a pharmaceutical composition. A “pharmaceutical composition” is a composition comprising a therapeutically effective amount of a nucleic acid or polypeptide. The terms “effective amount” and “therapeutically effective amount” each refer to the amount of a nucleic acid or polypeptide used to observe the desired therapeutic effect (e.g., induce or enhance an immune response).

Injectable preparations, such as sterile injectable aqueous or oleaginous suspensions, may be formulated according to known methods using suitable dispersing or wetting agents and suspending agents. The injectable preparation may also be a sterile injectable solution or suspension in a non-toxic parenterally acceptable diluent or solvent. Suitable vehicles and solvents that may be employed are water, Ringer's solution, and isotonic sodium chloride solution, among others. For instance, a viral vector such as a poxvirus may be prepared in 0.4% NaCl or a Tris-HCl buffer, with or without a suitable stabilizer such as lactoglutamate, and with or without freeze drying medium. In addition, sterile, fixed oils are conventionally employed as a solvent or suspending medium. For this purpose, any bland fixed oil may be employed, including synthetic mono- or diglycerides. In addition, fatty acids such as oleic acid find use in the preparation of injectables.

Pharmaceutical compositions may take any of several forms and may be administered by any of several routes. The compositions are administered via a parenteral route (e.g., intradermal, intramuscular, subcutaneous, skin scarification) to induce an immune response in the host. Alternatively, the composition may be administered directly into a tissue or organ such as nose, vagina, rectum, a lymph node (e.g., intranodal) or tumor mass (e.g., intratumoral). Preferred embodiments of administratable compositions include, for example, nucleic acids, viral particles, or polypeptides in liquid preparations such as suspensions, syrups, or elixirs. Preferred injectable preparations include, for example, nucleic acids or polypeptides suitable for parenteral, subcutaneous, intradermal, intramuscular or intravenous administration such as sterile suspensions or emulsions. Mucosally administered preparations may be mixed with a gel or be presented in freeze-dried tablets or in device for a sustained release of the immunogen (e.g., EN41-FPA2 with a gel). For example, a naked DNA molecule and/or recombinant poxvirus may separately or together be in admixture with a suitable carrier, diluent, or excipient such as sterile water, physiological saline, glucose or the like. The composition may also be provided in lyophilized form for reconstituting, for instance, in isotonic aqueous, saline buffer. In addition, the compositions can be co-administered or sequentially administered with one another, other antiviral compounds, other anti-cancer compounds and/or compounds that reduce or alleviate ill effects of such agents.

As previously mentioned, while the compositions described herein may be administered as the sole active agent, they can also be used in combination with one or more other compositions or agents (i.e., other immunogens, co-stimulatory molecules, adjuvants). When administered as a combination, the individual components can be formulated as separate compositions administered at the same time or different times, or the components can be combined as a single composition. In one embodiment, a method of administering to a host a first form of an immunogen and subsequently administering a second form of the immunogen, wherein the first and second forms are different, and wherein administration of the first form prior to administration of the second form enhances the immune response resulting from administration of the second form relative to administration of the second form alone, is provided. Also provided are compositions for administration to the host. For example, a two-part immunological composition where the first part of the composition comprises a first form of an immunogen and the second part comprises a second form of the immunogen, wherein the first and second parts are administered together or separately from one another such that administration of the first form enhances the immune response against the second form relative to administration of the second form alone, is provided. The immunogens, which may be the same or different, are preferably derived from the infectious agent or other source of immunogens. The multiple immunogens may be administered together or separately, as a single or multiple compositions, or in single or multiple recombinant vectors. For instance, a viral vector encoding an immunogen may be initially administered and followed by one or more subsequent administrations with a second form of the immunogen (e.g., a polypeptide). The different forms may differ in either or both of the form of delivery (e.g., viral vector, polypeptide) or in the immunogens represented by each form. It is preferred that the forms, however, induce or enhance the immune reponse against a particular target (e.g., HIV-1). Typically, both the priming and boosting doses are administered via the same route (e.g., intramuscular, intradermal, mucosal) but the routes of administration may also be different (e.g., priming via the intramuscular, intradermal, mucosal, and boosting via intramuscular, intradermal, mucosal where the routes of administration in the priming and boosting administrations are different). Typically, the priming and boosting doses are administered to different parts of the body, but the doses may also be administered to the same part of the body. “Along with” may mean that the two forms are administered as separate compositions, as part of a single composition, at separate sites of the body, or at the same site of the body, depending on the particular protocol. Variations of such exemplary dosing regimens may be made by those of skill in the art.

For example, a composition for immunizing a mammal against HIV (e.g., a vaccine) may comprise the gp41-derived protein, FP-UGR7-MPR-A-2 (SEQ ID NO.: 1) formulated in a liposome that also contains an adjuvant such as monophosphoryl lipid A (MPLA). In one embodiment, each mL of liposomal suspension (e.g., comprising DMPC, cholesterol, DMPG in a suitable molar ratio, buffer (e.g., PBS, PB-saccharose), and detergent (e.g., Tween-20, β-OG)) may contain 1 mg of FP-UGR7-MPR-A-2 and 800 μg of MPLA (e.g., “EN41-FPA2 suspension”). The mode of administration may include, for example, at least one nasal administration followed by at least one intra-muscular (IM) administration (e.g., an exemplary prime-boost protocol). For instance, an exemplary prime-boost immunization protocol may comprise one, two, three, four or five priming immunizations administred by the nasal route followed by one, two, three, four, or five booster immunizations by the intramuscular route (e.g., up to 28 days after the final nasal immunization). For nasal administration, a liposomal suspension comprising FP-UGR7-MPR-A-2 (SEQ ID NO.: 1) (e.g., EN41-FPA2) may be mixed at a suitable ratio (v/v) (e.g., about any of 0.5:1, 1:1, 1.5:1, 2:1) with another composition (e.g., HEC gel composition (4% w/w Natrosol HHX (Hydroxyethyl cellulose), 1.1% w/w Benzyl alcohol in PBS)). The mammal may receive an appropriate amount in one or both nostrils (e.g., 40 μL in a single nostril corresponding to 20 μg protein and 16 μg MPLA; 200 μL in a single nostril corresponding to 100 μg of protein and 80 μg MPLA; 200 μL in each nostril, i.e. 400 μL corresponding to 200 μg of protein and 160 μg MPLA). A control nasal administration may include the HEC gel composition only. For IM administration, a liposomal suspension comprising FP-UGR7-MPR-A-2 (SEQ ID NO.: 1) (e.g., EN41-FPA2) may be mixed with, for example, 0.9% NaCl (e.g., 400 μL of the diluted suspension, corresponding to 200 μg of protein and 160 μg MPLA). The control IM administration may include 0.9% NaCl only. An exemplary trial designed is described herein in the Examples section.

Any one or more of the following parameters may be measured to determine the safety and/or immunogenicity of these systems (e.g., among many others as would be understood by those of ordinary skill in the art): EN41-FPA-2 specific serum IgG responses by ELISA assay induced by the vaccine candidate (e.g., up to 28 days after the final immunisation); the neutralising activity against HIV in serum and vaginal samples using PBMC and TZM-b1 assays (e.g., up to 28 days after the final immunisation); neutralising activity against HIV in serum and vaginal samples using PBMC and TZM-b1 assays (e.g., up to 6 months after the final immunisation); specific B cell responses (e.g., as measured by ELISPOT assay); inhibitory activity measured by virus capture assay; inhibitory activity by ADCC assay using primary NK cells; Fc-mediated inhibitory activity on macrophages; inhibition of HIV transfer from DC to CD4+ T lymphocytes (e.g., by antibodies); T-cell responses as determined using Intracellular Cytokine Staining (ICS) using multi-parametric flow cytometry or ELISPOT assay; kinetics of the immune response; inhibitory antibody activity (neutralization, ADCC, Fc-mediated inhibition) using, for example, purified IgG/IgA from plasma; the proportion of individuals mounting a serum IgG response to EN41-FPA2 at any time point up to 28 days (or 6 months) after the final immunisation with a three-fold increase from pre-immunisation baseline sample taken at visit 2 (week 0, priming #1) (if no serum sample is available at this time point, serum taken at visit 1, screening may be substituted); proportion of individuals mounting a serum IgA response to EN41-FPA2; proportion of individuals mounting a mucosal antibody response to EN41-FPA2 (either IgG or IgA) in vaginal secretion samples; increase from baseline of neutralising activity in serum, vaginal and/or cervico-vaginal secretions against Tier 1 virus, measured as IC₈₀ in PBMC assays and IC₅₀ in TZM-b1 assays (e.g., “baseline” being the result of the sample taken at visit 2, week 0 (or in the case of serum at screening visit 1, if no sample is available from visit 2)), where a positive response is represented by a three-fold increase from baseline occurring any time after the first immunisation; comparison of serum ELISA IgG titers in the various groups (e.g., groups 3 and 4 in Table 9) to investigate the importance of priming in the immunization scheme; proportion of volunteers with specific B cell responses measured by ELISPOT assay; proportion of volunteers with inhibitory activity measured by virus capture assay; proportion of volunteers with inhibitory activity by ADCC assay; proportion of volunteers with Fc-mediated inhibitory activity; proportion of volunteers with inhibition of HIV transfer from DC to CD4⁺ T lymphocytes; proportion of volunteers mounting a T cell response measured by either ICS or ELISPOT assay; kinetics assessed by measurement of the ELISA response at all timepoints. Adverse events (e.g., grade 3 or above) may also be monitored to confirm safety. Other parameters may also be measured as would be understood by one of ordinary skill in the art.

Kits are also provided. For example, a kit comprising a composition described herein may be provided. The kit can include a separate container containing a suitable carrier, diluent or excipient. The kit may also include additional components for simultaneous or sequential-administration. In one embodiment, such a kit may include a first form of an immunogen and a second form of the immunogen. Additionally, the kit can include instructions for mixing or combining ingredients and/or administration. A kit may provide reagents for performing screening assays, such as one or more PCR primers, hybridization probes, and/or biochips, for example. For example, an exemplary kit may include one or more compositions and/or reagents (and/or reagents for preparing the same) for immunizing a mammal against HIV (e.g., a vaccine). One composition may comprise a gp41-derived protein, such as FP-UGR7-MPR-A-2 (SEQ ID NO.: 1), and another may be a liposomal composition that may be mixed with the protein to produce a formulation for immunizing a mammal (e.g., a liposome comprising the protein and an adjuvant such as monophosphoryl lipid A (MPLA)). The kit may also include additional compositions and/or reagents that may assist the user in carrying out a particular immunization scheme (e.g., preparing a mixture of the liposomal formulation with an HEC gel composition (4% w/w Natrosol HHX (Hydroxyethyl cellulose), 1.1% w/w Benzyl alcohol in PBS) for nasal administration, or with 0.9% NaCl for intramuscular (IM) administration). Direction for setting up and/or carrying out an immunization protocol may also be included (e.g., instructing the user regarding intranasal and/or intramuscula administration, either alone or as part of a prime-boost protocol). For instance, the instructions may instruct the user to carry out a prime-boost immunization protocol by administering one, two, three, four or five priming immunizations administred by the nasal route followed by one, two, three, four, or five booster immunizations by the intramuscular route at a particular time points (e.g., begin IM administration(s) within 28 days after the final nasal immunization). In one embodiment, the instructions may assist the user in setting up an experimental trial, as described in in the Examples section (e.g., Table 9), which may of course be modified to fit the particular formulation(s) being tested by the user. The kit may also include any one or more of the reagens required to measure the safety and/or immunogenicity of the formulations/systems described herein. Other embodiments of kits are also contemplated herein as would be understood by one of ordinary skill in the art.

A better understanding of this disclosure and of its many advantages may be deduced from the following examples, given by way of illustration.

### EXAMPLES

### Example 1

### Gp41 Polypeptides and Nucleic Acids

An exemplary modified gp41 polypeptide was prepared as described below. The amino acid sequence of the modified gp41 polypeptide was based on the gp41 ectodomain of HIV LAI (Swiss-Prot entry P03377) (e.g., FIG. 1). The ectodomain was used as a stable scaffold to display the MPER region in a trimeric arrangement. The Polar Region (“PR”; AGSTMGARSMTLTVQA (SEQ ID NO.:3)) may act to occlude the 2F5 and 4E10 epitopes and was not included in the construct. The Fusion Peptide region (FP; amino acids 1-13 as in FIG. 2) was thereby positioned adjacent the MPER region, which is believed to stabilize the latter in an immunogenic conformation. The modified gp41 polypeptide (SEQ ID NO.:1) included both the 2F5 (ELDKWAS; SEQ ID NO.: 4) and 4E10 (NWFNIT; SEQ ID NO.: 5) epitopes. The C-terminal residues NWLW (SEQ ID NO.: 6) were included to facilitate incorporation of the modified gp41 polypeptide into liposomes. An N-terminal expression tag (MHKVHGSGSGS (SEQ ID NO.: 2) was included to facilitate expression in E. coli.

Seven mutations to the ectodomain were included in the modified gp41 polypeptide to reduce hydrophobicity, reduce β-sheet propensity, and increase the net charge of the molecule (e.g., reduce the isoelectric point). W85 is solvent-exposed, bulky and hydrophobic, and modification thereof was hypothesized to reduce hydrophobicity, increase net charge and helical propensity. I92 is also solvent-exposed, and was selected for reasons similar to W85. The modification of A96 (e.g., A96E) was hypothesized to increase the net charge at a solvent-exposed position. The T95P substitution was selected because T95 is oriented toward the interior of the loop and has a main-chain conformation compatible with proline, and proline is a known beta-sheet “breaker”. Previous work by Krell, et al. (EJB 271, 1566 (2004)) showed a moderate solubility increase in the gp41 (S30) triple mutant containing L91K, 192K, and W103D (soluble up to 0.08 mg/mL in 10 mM Na₂HPO₄/NaH₂PO₄, 0.05% Tween-20, pH 7.5). These amino acids were also selected for substitution. Amino acid Leu81 was substituted due to its high solvent exposure and hydrophobicity. The amino acid substitions ulitimately made in this modified gp41 polypeptide were L81D, W85E, L91G, 192D, T95P, A96E, and W103D. The amino acid sequence of this modified gp41 polypeptide (“FP-UGR7-MPR-A-2”) is shown below:

(SEQ ID NO.: 1; FIG. 2)
M H K V H G S G S G S A V G I G A L F L G F L G A
R Q L L S G I V Q Q Q N N L L R A I E A Q Q H L L
Q L T V W G I K Q L Q A R I L A V E R Y L K D Q Q
D L G I E G C S G K G D C T P E V P W N A S D S N
K S L E Q I W N N M T W M E W D R E I N N Y T S L
I H S L I E E S Q N Q Q E K N E Q E L L E L D K W
A S L W N W F N I T N W L W

FIG. 1 provides an alignment 132 HIV-1 gp41 sequences, indicating for each residue its level of conservation. Mutations introduced in FP-UGR7-MPR-A-2 are circled. Of the seven substitions made to gp41, two are at completely conserved residues and three are at residues conserved between 80-99% of the 132 HIV-1 gp41 sequences analyzed. This high level of amino acid conservation of FP-UGR7-MPR-A-2 indicates that the modified polypeptide is likely to induce an immune response against many different subtypes of HIV-1.

The nucleic acid construct encoding FP-UGR7-MPR-A-2 was used to express the polypeptide (SEQ ID NO.: 1) in E. coli. The strain used for FP-UGR7-MPR-A-2 expression is BLR(DE3) (Novagen, reference: 69053). Other strains with DE3 prophage could be used (e.g., BL21(DE3)). The plasmid used for expression is PM1800 provided by SP. The sequence of the booster peptide is the following:

(SEQ ID NO.: 7)
atg cat aaa gtt cat ggt agc ggt agc ggc agc
(SEQ ID NO.: 2)
M H K V H G S G S G S

This peptide was found to greatly increase the level of expression of the recombinant polypeptide, FP-UGR7-MPR-A-2 in a prokaryotic expression system. One skilled in the art can appreciate any suitable expression plasmid can be used to express the recombinant FP-UGR7-MPR-A-2 polypeptide in the suitable prokaryotic or bacterial host.

The recombinant polypeptide was found to exhibit high solubility (at least 0.6 mg/mL in 50 mM sodium phosphate) at physiological pH as intended in its design. The hydrodynamic radius (Rh) measured by dynamic light scattering indicates that the recombinant polypeptide is trimeric at pH 2.5 and oligomeric at pH 7.4 (Table 2).

TABLE 2

| Sample | pH 2.5, Rh (nm) | pH 7.4, Rh (nm) |
|---|---|---|
| FP-UGR7MPR-A-2 | 4.2 (90%), 9.5 (10%) | 18.3 (85%), 47 (15%) |

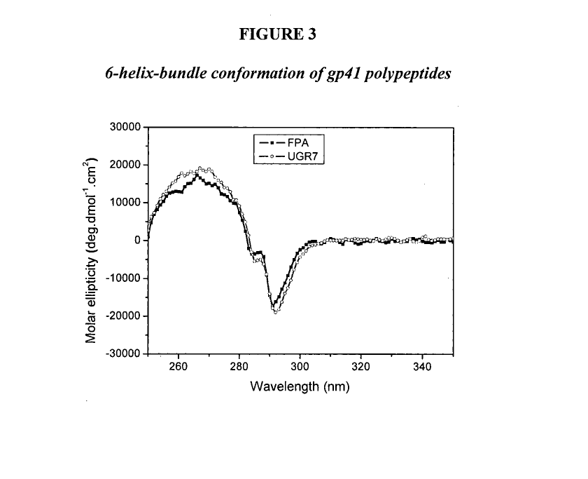

Circular dichroism (CD) was used to determine the protein conformation. The far- and near-UV CD at both pH 2.5 and 7.4 were measured and compared with those of a shorter gp41 polypeptide (named UGR7), which lacks the fusion peptide sequence AVGIGALFLGFLG (SEQ ID NO: 8) and the membrane proximal sequence LWNWFNITNWLW (SEQ ID NO.: 9). It was determined that the α-helical content is high in both proteins (Table 3), with about 10 additional residues in α-helical conformation for FP-UGR7-MPR-A-2 as compared to UGR7. The near UV-CD data (FIG. 3) indicates that the two polypeptides adopt a similar overall fold consisting of a trimer of hairpins in a 6-helix-bundle conformation, as observed for other recombinant polypeptides of the gp41 ectodomain (Peisajovich et al. 2003 J. Mol. Biol. 326, 1489-1501).

TABLE 3

| | | | [θ]_{MRW} | | Residues |
|---|---|---|---|---|---|
| Antigen | pH | Residues | (222 nm) | % α-helix | in α-helix |
| UGR7 | 2.5 | 145 | −24236 | 68.0 | 99 |
| UGR7 | 7.4 | 145 | −23793 | 66.8 | 97 |
| FP-UGR7-MPR-A-2 | 2.5 | 175 | −22681 | 63.5 | 111 |
| FP-UGR7-MPR-A-2 | 7.4 | 175 | −21706 | 60.9 | 106 |

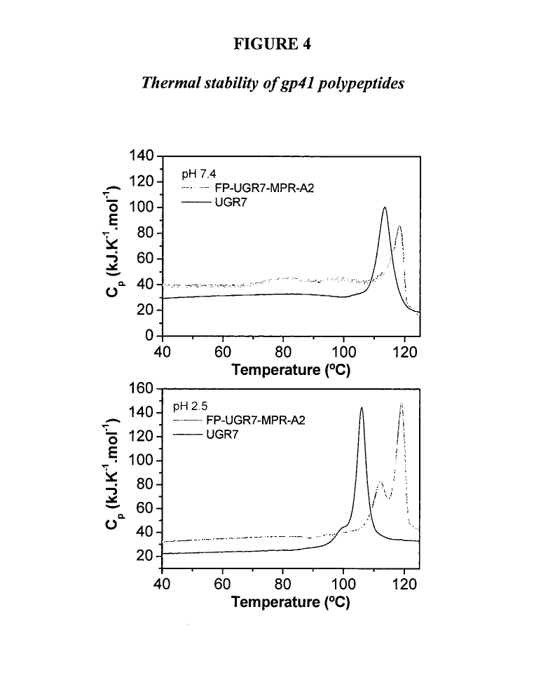

It was also found to exhibit high thermal stability, as measured by differential scanning calorimetry (FIG. 4). The data illustrates that the thermal denaturations were partially reversible at pH 2.5 and completely irreversible at pH 7.4. The presence of the FP and the MPR regions in close proximity to each other increases the stability of FP-UGR7-MPR-A-2 as compared to that of UGR7, which lacks those regions.

In summary, this data shows that: 1) FP-UGR7-MPR-A-2 is soluble and essentially oligomeric at neutral pH; 2) the alpha-helical content is high with an overall fold of a trimer of hairpins in a 6-helix-bundle conformation; 3) this conformation facilitates the close contact between the FP and MPR regions, resulting in additional structure; 4) FP-UGR7-MPR-A-2 shows a high thermal stability; and, 5) the contact between the FP and MPR regions appears to considerably stabilize the molecule.

### Example 2

### Liposomal Formulations

The antigen design (e.g., formulations) should provide for presentation of the MPER region in the context of a lipid environment (with or without an additional adjuvant). Several reports in the literature have indicated that the broadly neutralizing mAbs 2F5 and 4E10 recognize their putative epitopes in a lipid environment with a much higher avidity than in solution. Thus, the FP-UGR7-MPR-A-2-polypeptide was prepared in a liposomal composition to produce the composition “EN41-FPA2”. It was determined that the liposomes should consist of DMPC, cholesterol and DMPG in a molar ratio of 9:1:7, and include the adjuvant MPLA (a TLR4 agonist) (tested at various concentrations). MPLA is co-solubilized together with the lipids in ethanol. MPLA concentration in the liposomes ranged between 0.1-1 mg/mL. To allow sterile filtration of the final product, the liposomes were to exhibit a z-average mean of 80-130 nm and polydispersity index below about 0.25.

### A. Materials and Methods

The phospholipids di-myristoyl-phosphatidylcholine (DMPC) and di-myristoyl-phosphatidylglycerol (DMPG) were purchased from Lipoid (Switzerland) whereas cholesterol was obtained from Solvay Pharmaceuticals (Netherlands). Liposomes consisting of DMPC, cholesterol and DMPG in the molar ratio of 9:1:7 were prepared. The lipid components were used in the same molar ratio for most of the experiments but at varying start concentrations. The gp41 polypeptide FP-UGR7-MPR-A-2 (SEQ ID NO.: 1) was supplied in 50 mM phosphate buffer pH 7.5 containing 0.014-0.0014% Tween 20. Throughout all experiments, the lipids were dissolved in 96% ethanol (Merck). The final ethanol concentration in the aqueous phase ranged between 7.5 and 10%. The buffers and aqueous phases used in the experiments where PBS pH 7.4 and PB-Saccharose pH 7.5 with one of β-OG, Chaps or Tween 20 for micellar membrane protein dissolution. The experiments were performed either at room temperature or 55° C. The ethanolic lipid solution has to be tempered at least 55° C. independent of the temperature of the aqueous phase in order to obtain lipid solubilization.

Liposomes were produced by crossflow injection. A continuous aseptic one step operation permits the production of stable and sterile liposomes with a defined size distribution. The production equipment was designed to meet several requirements including simplicity, robustness and easy handling in sterilization procedures. The injection modules used in the experiments were equipped with 250 μm and 350 μm injection whole diameters. Using these systems, increased amounts of lipid ethanol can be injected into the aqueous phase by dilution of the liposome suspension immediately after injection without any side effects concerning membrane stability. This process step increases passive encapsulation rates significantly. The volume of the dilution buffer was varied throughout the optimization procedure resulting in increased ethanol concentration of the intermediate liposome solution.

As prime analytical marker, measurements for the determination of liposome size were performed by Dynamic Laser Light Scattering with a Malvern Nano ZS. This system is equipped with a 4 mW Helium/Neon laser at 633 nm wavelength and measures the liposome samples with the non-invasive backscatter technology at a detection angle of 173°. The experiments were carried out at 25° C. The results are presented in an average diameter of the liposome suspension (z-average mean) with the polydispersity index (PdI) to determine liposome homogeneity.

The average amount of encapsulated and non-entrapped gp41 polypeptide (FP-UGR7-MPR-A-2) was determined by SDS-PAGE on a Novex system and by reverse phase (RP)—HPLC. For the determination of the incorporated protein content, the liposome sample was separated from non-entrapped protein by diafiltration. The membrane-incorporated gp41 (FP-UGR7-MPR-A-2) was determined in the retentate and the non-entrapped protein was quantified in the filtrate. Filtered liposome sample (retentate) and unbound protein (filtrate) was spotted onto an electrophoresis gel (e.g., NOVEX Tris/Glycine gel). For protein specificity testing, the gel was electroblotted onto PVDF Immobilon P 0.45 μM (Millipore) for 2 hours and viral membrane antigens specifically stained with hmAb 2F5 and visualized using anti-human IgG conjugated with alkaline phosphatase. Additionally, samples were examined with respect to pH, with respect to osmolality and with respect to zeta potential.

### B. Experiments and Results

Study 1. Empty Liposome Formulation with Polymun's Standard Conditions

In the first set of experiments, empty liposomes were performed without addition of MPLA. The experiments were performed using standard conditions, which were developed in previous projects and are listed in Table 4.

TABLE 4
Process parameters of Study 1

| | |
|---|---|
| Aqueous phase | PBS pH 7.4 and PB-Saccharose |
| | pH 7.5 with one of 1% β-OG, |
| | 0.014% Tween 20, or 1.2% Chaps |
| Injection module diameter | 250 μm |
| Ethanol concentration in intermediate | 7.5% |
| liposome suspension | |
| Volume ratio injection/dilution buffer | 20 ml/80 ml |
| Temperature ethanol solution | 55° C. |
| Temperature aqueous phases | 55° C. |

The experiments started with a lipid concentration of 5 μmol/ml aqueous phase (a total of 504.8 μmol lipids were dissolved in 7.5 ml ethanol which was heated and stirred for dissolution of lipids) as an ethanolic lipid solution. This ethanolic lipid solution was injected into 20 ml PBS containing a detergent in appropriate concentration (see Table 5) that was simultaneously diluted with additional 80 ml PBS. The results are summarized in Table 5.

TABLE 5
Summary of data from liposomes of Study 1

| | DMPC | | | | | |
|---|---|---|---|---|---|---|
| Experi- | (μmol/ | Chol | DMPG | | z-average | |
| ment | ml) | (μmol/ml) | (μmol/ml) | Detergent | mean (nm) | PdI |
| 1 | 2.7 | 2.1 | 0.3 | 1% | 120.6 | 0.143 |
| | | | | β-OG | | |
| 2 | 2.7 | 2.1 | 0.3 | 0.014% | 79.4 | 0.196 |
| | | | | Tween-20 | | |
| 3 | 2.7 | 2.1 | 0.3 | 1.2% | 400.3 | 0.511 |
| | | | | Chaps | | |

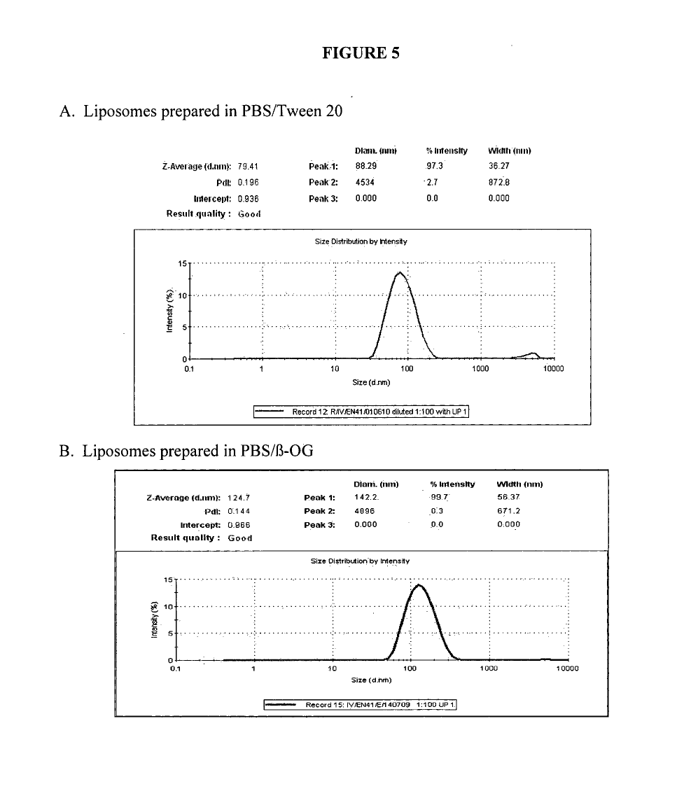

This data is representative of several studies. The data showed consistently well-defined and homogeneous liposomes (e.g., as indicated by the z-average mean), if prepared in presence of PBS/β-OG or PBS/Tween-20, whereas the formulations in presence of Chaps were consistently large and heterogeneous, independent of the process parameters, which were applied (FIG. 5).

Part 2. Liposome Formulation with FP-UGR/-MPR-A-2

The process parameters for the preparation of liposomal associated gp41 constructs are as described above. The gp41 polypeptide FP-UGR7-MPR-A-2 (SEQ ID NO.: 1) was provided in 50 mM phosphate buffer/0.01464% Tween-20. After thawing the protein samples, the protein solutions were diluted with a batch specific detergent containing PB-Saccharose buffer (Na₂HPO₄*2H₂O (1.44 g/L), KCl (0.2 g/L), KH₂PO₄ (0.2 g/L), saccharose (92.42 g/L) and 0.0146% Tween 20) to a concentration of 0.25-0.30 mg/ml. All other process parameters are given in Table 6.

TABLE 6
Description of process parameters for experimental part 2

| | |
|---|---|
| Aqueous phases batch 1 | PB-Saccharose/0.01464% |
| | Tween 20 |
| Injection module diameter | 250 μm |
| Ethanol concentration in intermediate | 7.5% |
| liposome suspension | |
| Volume ratio injection/dilution buffer | 20 ml/80 ml |
| Temperature ethanol solution | 55° C. |
| Temperature aqueous phases | 55° C. |

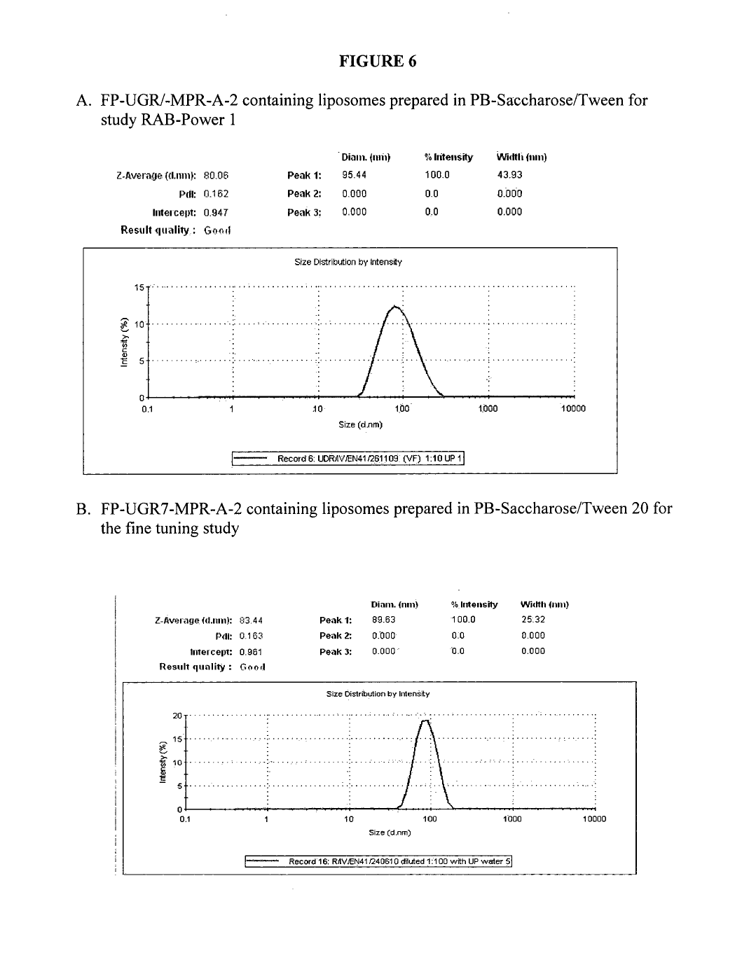

The resulting liposome suspensions were in the size range of 80 nm-90 nm and very homogeneous, as indicated by a PdI <0.25. FIGS. 6A and 6B show representative samples, which were used for animal studies.

Besides size measurements, the samples were additionally analyzed by electrophoresis and Western blot which indicates the recombinant gp41 constructs were inserted into the liposomal membranes by this process. Filtrates were also assessed and showed no free gp41. Thus, it was concluded that essentially all of the gp41 polypeptide was inserted into the liposomes membranes. Similar data were observed in another experiment.

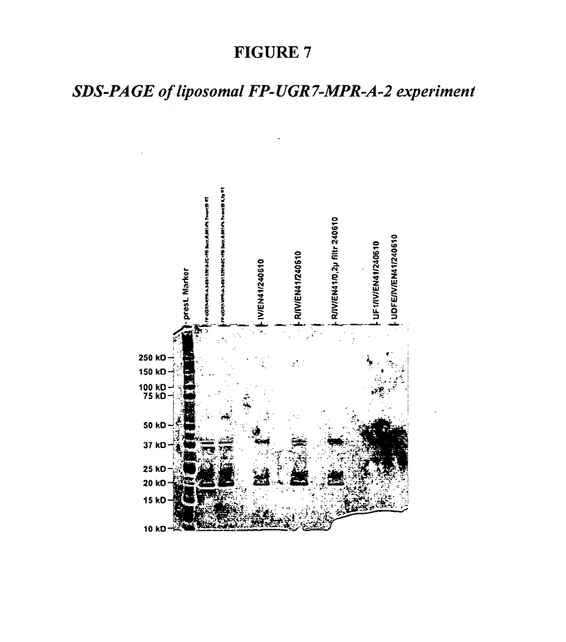

Thus, a new technique for the efficient association of membrane proteins with liposomal membrane systems by combining an advanced ethanol injection technique—the crossflow injection technique—with detergent dilution within one operational step, has been developed. Using this method, membrane proteins are inserted into uniform liposomes under controlled conditions with the possibility of fast, aseptic production in industrial scale. The data indicates that PBS/octyl-β-D-glucopyranoside (PBS/β-OG) or PBS/Tween 20 support the preparation of small and homogeneous distributed liposomes. In contrast, where Chaps was used, liposomes composed of DMPC, DMPG, cholesterol and MPLA became very large and heterogeneous. The data indicates that homogeneously distributed proteoliposomes with vesicle size in the range of 70-130 nm are formed in the presence of β-OG or Tween 20. The samples were treated in a filtration unit equipped with a 100 kDa PES-membrane to determine the amount of non-entrapped gp41 and to remove detergent and ethanol, necessary for proteoliposome formation. These samples were analyzed by electrophoreses and Western blot. The data indicated that, independent of the generated vesicle sizes, almost 100% of the admitted protein was entrapped within the liposomes. Gp41 polypeptide was not detected in the filtrate lanes (the maximum protein mass is about 60 kD assuming a trimeric configuration) (FIG. 7). In addition, the data indicates that the added protein is neither denatured nor destroyed during the preparation procedure. The protein bands are similarly independent of thermal, chemical and mechanical influences during liposome formulation. This is of particular interest, because locally high ethanol concentrations are generated at the injection site which in combination with injection temperatures around 55° C. might have damaged the biologic material. Thus, the gp41 polypeptide solubilized in Tween-20 or β-OG may be used to prepare liposomes of a uniform size range. These liposomes may then be subjected to a sterile filtration process prior to use.

### Example 3

### Immunogenicity of FP-UGR7-MPR-A-2

### A. Non-Human Animal Studies

The gp41 polypeptide liposomal compositions described in Example 2 were tested for immunogenicity as described herein. Various routes of immunization (intramuscular, mucosal (vaginal, nasal, sublingual)) were tested in rabbits. Various adjuvants were also tested including alum, IMS, CT/Alum, and MPLA. Various formulations were also tested including drops, rods, and tablets. Various dosing schedules were also tested including close versus remote re-immunization and prime-boost protocols. A positive result was determined by ELISA, or using a neutralization (TZMb1 assay: IC₅₀ at 1/40 dilution for sera, IC₅₀ at 1/16 dilution for lavages; PBMC assay: IC₈₀ at ¼ dilution for sera and lavages) and/or Fc-gamma mediated macrophage inhibition (IC₈₀ at ¼ dilution for sera and lavages) assays. The formulated gp41 polypeptides that were tested included FPA (“FP-UGR7-MPR-A-2; SEQ ID NO.: 1), PR-UGR7-MPR-A (“PRA”), FP-UGR7-MPR-B (“FPB”), and 4B1C. The data resulting from immunization experiments is summarized in Tables 7 and 8.

TABLE 7
RAB-POWER-1:- FPA, PR-A, FP-B, Harvard 4B1C in liposomes + MPLA: IM, IN, Ivag

| BDG/ | | | | ROUTE OF | | | Prime | Prime | | Prime | | |
|---|---|---|---|---|---|---|---|---|---|---|---|---|
| NEUT | SPLE | Ag | GROUP | ADMIN. | Test | D-7 | D0 | D14 | D21 | D28 | D35 | D42 |
| BINDING | SERUM | FP-UGR7- | Group 1 | IM | IgG | NS | − | NS | ++ | | +++ | |
| RESULTS | | MPR-A | (R310-314) | | IgA | | − | | | | ++ | |
| | | | Group 2 | NASAL | IgG | NS | − | NS | | | − | |
| | | | (R315-319) | | IgA | | − | | | | − | |
| | | | Group 3 | VAGINAL | IgG | NS | − | NS | | | ++ | |
| | | | (R320-324) | | IgA | | − | | | | − | |
| | | PR-A | Group 4 | IM | IgG | NS | − | NS | | | +++ | |
| | | | (R325-329) | | IgA | | − | | | | + | |
| | | FP-UGR7- | Group 5 | IM | IgG | NS | − | NS | | | +++ | |
| | | MPR-B | (R330-334) | | IgA | | − | | | | ++ | |
| | | | Group 6 | NASAL | IgG | NS | − | NS | | | − | |
| | | | (R335-339) | | IgA | | − | | | | − | |
| | | | Group 7 | VAGINAL | IgG | NS | − | NS | | | +++ | |
| | | | (R340-344) | | IgA | | − | | | | + | |
| | | 4B1C | Group 8 | IM | IgG | NS | − | NS | | | +++ | |
| | | | (R345-349) | | IgA | | − | | | | ++ | |
| | | | Group 9 | NASAL | IgG | NS | − | NS | | | − | |
| | | | (R350-354) | | IgA | | − | | | | − | |
| | | | Group 10 | VAGINAL | IgG | NS | − | NS | | | + | |
| | | | (R355-359) | | IgA | | − | | | | − | |
| | | Controls | Group 11 | IM | IgG | NS | − | NS | | | − | |
| | | | (R360-364) | | IgA | | − | | | | − | |
| | | | Group 12 | NASAL | IgG | NS | − | NS | | | − | |
| | | | (R365-369) | | IgA | | − | | | | − | |
| | | | Group 13 | VAGINAL | IgG | NS | − | NS | | | − | |
| | | | (R370-374) | | IgA | | − | | | | − | |
| | | STNLS | Group 14 | SENTINELS | IgG | NS | − | NS | | | − | |
| | | | (R375-379) | | IgA | | − | | | | − | |
| | LAVAGES | FP-UGR7- | Group 1 | IM | IgG | − | NS | NS | | NS | − | |
| | | MPR-A | (R310-314) | | IgA | + | | | | | + | |
| | | | Group 2 | NASAL | IgG | − | NS | NS | NS | NS | − | |
| | | | (R315-319) | | IgA | − | | | | | − | |
| | | | Group 3 | VAGINAL | IgG | − | NS | NS | NS | NS | − | |
| | | | (R320-324) | | IgA | − | | | | | + | |
| | | PR-A | Group 4 | IM | IgG | − | NS | NS | | NS | − | |
| | | | (R325-329) | | IgA | + | | | | | − | |
| | | FP-UGR7- | Group 5 | IM | IgG | − | NS | NS | | NS | − | |
| | | MPR-B | (R330-334) | | IgA | + | | | | | + | |
| | | | Group 6 | NASAL | IgG | − | NS | NS | NS | NS | − | |
| | | | (R335-339) | | IgA | + | | | | | + | |
| | | | Group 7 | VAGINAL | IgG | − | NS | NS | NS | NS | − | |
| | | | (R340-344) | | IgA | − | | | | | + | |
| | | 4B1C | Group 8 | IM | IgG | − | NS | NS | | NS | − | |
| | | | (R345-349) | | IgA | − | | | | | − | |
| | | | Group 9 | NASAL | IgG | − | NS | NS | NS | NS | − | |
| | | | (R350-354) | | IgA | − | | | | | − | |
| | | | Group 10 | VAGINAL | IgG | − | NS | NS | NS | NS | − | |
| | | | (R355-359) | | IgA | − | | | | | − | |
| | | Controls | Group 11 | IM | IgG | − | NS | NS | | NS | − | |
| | | | (R360-364) | | IgA | − | | | | | + | |
| | | | Group 12 | NASAL | IgG | − | NS | NS | NS | NS | − | |
| | | | (R365-369) | | IgA | − | | | | | + | |
| | | | Group 13 | VAGINAL | IgG | − | NS | NS | NS | NS | − | |
| | | | (R370-374) | | IgA | + | | | | | + | |
| | | STNLS | Group 14 | SENTINELS | IgG | − | NS | NS | | NS | − | |
| | | | (R375-379) | | IgA | − | | | | | − | |

| BDG/ | | | | ROUTE OF | | IM | IM | | | We-C | We-C |
|---|---|---|---|---|---|---|---|---|---|---|---|
| NEUT | SPLE | Ag | GROUP | ADMIN. | Test | D56 | D70 | D77 | D84 | Vag. | Vest. |
| BINDING | SERUM | FP-UGR7- | Group 1 | IM | IgG | +++ | +++ | +++ | +++ | | |
| RESULTS | | MPR-A | (R310-314) | | IgA | + | + | | + | | |
| | | | Group 2 | NASAL | IgG | − | + | + | + | | |
| | | | (R315-319) | | IgA | − | − | | + | | |
| | | | Group 3 | VAGINAL | IgG | ++ | +++ | +++ | +++ | | |
| | | | (R320-324) | | IgA | − | − | | − | | |
| | | PR-A | Group 4 | IM | IgG | +++ | +++ | | +++ | | |
| | | | (R325-329) | | IgA | − | − | | − | | |
| | | FP-UGR7- | Group 5 | IM | IgG | +++ | +++ | | +++ | | |
| | | MPR-B | (R330-334) | | IgA | + | + | | + | | |
| | | | Group 6 | NASAL | IgG | − | − | | − | | |
| | | | (R335-339) | | IgA | − | − | | − | | |
| | | | Group 7 | VAGINAL | IgG | ++ | +++ | | +++ | | |
| | | | (R340-344) | | IgA | − | − | | + | | |
| | | 4B1C | Group 8 | IM | IgG | +++ | +++ | | +++ | | |
| | | | (R345-349) | | IgA | − | − | | + | | |
| | | | Group 9 | NASAL | IgG | − | − | | + | | |
| | | | (R350-354) | | IgA | − | − | | − | | |
| | | | Group 10 | VAGINAL | IgG | + | +++ | | +++ | | |
| | | | (R355-359) | | IgA | − | + | | − | | |
| | | Controls | Group 11 | IM | IgG | − | − | | − | | |
| | | | (R360-364) | | IgA | − | − | | − | | |
| | | | Group 12 | NASAL | IgG | − | − | | − | | |
| | | | (R365-369) | | IgA | − | − | | − | | |
| | | | Group 13 | VAGINAL | IgG | + | + | | + | | |
| | | | (R370-374) | | IgA | − | − | | − | | |
| | | STNLS | Group 14 | SENTINELS | IgG | − | − | | − | | |
| | | | (R375-379) | | IgA | + | + | | + | | |
| | LAVAGES | FP-UGR7- | Group 1 | IM | IgG | NS | − | | + | +++ | ++ |
| | | MPR-A | (R310-314) | | IgA | | + | | +++ | +++ | +++ |
| | | | Group 2 | NASAL | IgG | NS | − | | − | − | − |
| | | | (R315-319) | | IgA | | + | | ++ | +++ | +++ |
| | | | Group 3 | VAGINAL | IgG | NS | − | | − | +++ | + |
| | | | (R320-324) | | IgA | | +++ | | +++ | +++ | +++ |
| | | PR-A | Group 4 | IM | IgG | NS | − | | − | +++ | +++ |
| | | | (R325-329) | | IgA | | − | | + | +++ | +++ |
| | | FP-UGR7- | Group 5 | IM | IgG | NS | − | | − | +++ | +++ |
| | | MPR-B | (R330-334) | | IgA | | + | | +++ | +++ | ++ |
| | | | Group 6 | NASAL | IgG | NS | − | | − | − | − |
| | | | (R335-339) | | IgA | | + | | ++ | +++ | ++ |
| | | | Group 7 | VAGINAL | IgG | NS | − | | − | +++ | + |
| | | | (R340-344) | | IgA | | − | | +++ | +++ | +++ |
| | | 4B1C | Group 8 | IM | IgG | NS | − | | + | +++ | ++ |
| | | | (R345-349) | | IgA | | − | | + | + | ++ |
| | | | Group 9 | NASAL | IgG | NS | − | | − | − | − |
| | | | (R350-354) | | IgA | | − | | + | + | + |
| | | | Group 10 | VAGINAL | IgG | NS | − | | − | + | − |
| | | | (R355-359) | | IgA | | − | | − | +++ | + |
| | | Controls | Group 11 | IM | IgG | NS | − | | − | − | − |
| | | | (R360-364) | | IgA | | + | | + | +/− | +/− |
| | | | Group 12 | NASAL | IgG | NS | − | | − | − | +/− |
| | | | (R365-369) | | IgA | | + | | + | +/− | + |
| | | | Group 13 | VAGINAL | IgG | NS | − | | − | − | − |
| | | | (R370-374) | | IgA | | +++ | | + | +/− | − |
| | | STNLS | Group 14 | SENTINELS | IgG | NS | − | | − | | |
| | | | (R375-379) | | IgA | | − | | − | | |

KEYS:
NS: no sampling this day [symbol]
Negative result [symbol]
+for 1 or 2 rabbits [symbol]
+for 3 or 4 rabbits [symbol]
+for 5 rabbits [symbol]
Unclear result

TABLE 8
RAB-POWER-1:- FPA, PR-A, FP-B, Harvard 4B1C in liposomes + MPLA: IM, IN, Ivag

| BDG/ | | | | ROUTE | | | Prime | Prime | | Prime | | | IM | IM | | | We-C | We-C |
|---|---|---|---|---|---|---|---|---|---|---|---|---|---|---|---|---|---|---|
| NEUT | SPLE | Ag | GROUP | OF ADMIN. | Test | D-7 | D0 | D14 | D21 | D28 | D35 | D42 | D56 | D70 | D77 | D84 | Vag. | Vest. |
| NEUTRALISATION RESULTS | SERUM | FP-UGR7-MPR-A | Group 1 | IM | TZM | NS | − | NS | | | − | | | ++ | − | − | | |
| | | | (R310-314) | | PBMC | | + | | + | | ++ | | | ++ | + | − | | |
| | | | Group 2 | NASAL | TZM | NS | − | NS | | | − | | | +++ | + | − | | |
| | | | (R315-319) | | PBMC | | + | | − | | + | | | ++ | ++ | − | | |
| | | | Group 3 | VAGINAL | TZM | NS | − | NS | | | − | | | +++ | − | − | | |
| | | | (R320-324) | | PBMC | | ++ | | − | | ++ | | | ++ | + | − | | |
| | | PR-A | Group 4 | IM | TZM | NS | − | NS | | | − | | | +++ | + | − | | |
| | | | (R325-329) | | PBMC | | ++ | | | | +++ | | | ++ | + | − | | |
| | | FP-UGR7-MPR-B | Group 5 | IM | TZM | NS | − | NS | | | − | | | +++ | − | + | | |
| | | | (R330-334) | | PBMC | | + | | | | − | | | + | − | − | | |
| | | | Group 6 | NASAL | TZM | NS | − | NS | | | − | | | +++ | − | − | | |
| | | | (R335-339) | | PBMC | | − | | | | − | | | − | − | − | | |
| | | | Group 7 | VAGINAL | TZM | NS | − | NS | | | − | | | +++ | − | − | | |
| | | | (R340-344) | | PBMC | | + | | | | − | | | + | + | + | | |
| | | 4B1C | Group 8 | IM | TZM | NS | − | NS | | | − | | | +++ | − | − | | |
| | | | (R345-349) | | PBMC | | − | | | | − | | − | + | − | − | | |
| | | | Group 9 | NASAL | TZM | NS | − | NS | | | − | | | +++ | + | + | | |
| | | | (R350-354) | | PBMC | | − | | | | + | | + | + | + | + | | |
| | | | Group 10 | VAGINAL | TZM | NS | − | NS | | | ++ | | | ++ | − | − | | |
| | | | (R355-359) | | PBMC | | − | | | | + | | + | + | − | − | | |
| | | Controls | Group 11 | IM | TZM | NS | − | NS | | | + | | | + | − | − | | |
| | | | (R360-364) | | PBMC | | − | | | | + | | | + | − | − | | |
| | | | Group 12 | NASAL | TZM | NS | NS | NS | NS | | + | | | + | − | + | | |
| | | | (R365-369) | | PBMC | | + | | | | + | | | − | − | − | | |
| | | | Group 13 | VAGINAL | TZM | NS | − | NS | | | + | | | + | − | + | | |
| | | | (R370-374) | | PBMC | | − | | | | − | | | − | + | + | | |
| | | STNLS | Group 14 | SENTINELS | TZM | NS | − | NS | | | − | | | − | | − | | |
| | | | (R375-379) | | PBMC | | − | | | | − | | | − | | + | | |
| | LAVAGES | FP-UGR7- | Group 1 | IM | TZM | − | NS | NS | − | NS | − | | NS | − | − | − | − | − |
| | | MPR-A | (R310-314) | | PBMC | − | | | − | | − | − | | − | | + | +++ | + |
| | | | Group 2 | NASAL | TZM | − | NS | NS | NS | NS | − | | NS | − | − | − | − | − |
| | | | (R315-319) | | PBMC | − | | | | | − | − | | − | | − | +++ | + |
| | | | Group 3 | VAGINAL | TZM | − | NS | NS | NS | NS | − | | NS | − | − | − | − | − |
| | | | (R320-324) | | PBMC | − | | | | | − | − | | − | | − | +++ | − |
| | | PR-A | Group 4 | IM | TZM | − | NS | NS | − | NS | − | | NS | − | | − | − | |
| | | | (R325-329) | | PBMC | − | | | − | | + | | | − | | − | +++ | + |
| | | FP-UGR7- | Group 5 | IM | TZM | − | NS | NS | | NS | − | | NS | − | | − | − | |
| | | MPR-B | (R330-334) | | PBMC | + | | | | | − | | | − | | − | + | + |
| | | | Group 6 | NASAL | TZM | − | NS | NS | NS | NS | − | | NS | − | | − | − | − |
| | | | (R335-339) | | PBMC | − | | | | | − | | | − | | − | + | + |
| | | | Group 7 | VAGINAL | TZM | − | NS | NS | NS | NS | − | | NS | − | | − | − | − |
| | | | (R340-344) | | PBMC | − | | | | | − | | | + | | + | + | + |
| | | 4B1C | Group 8 | IM | TZM | − | NS | NS | | NS | − | | NS | − | | − | − | − |
| | | | (R345-349) | | PBMC | − | | | | | − | | | − | | − | − | − |
| | | | Group 9 | NASAL | TZM | − | NS | NS | NS | NS | − | | NS | − | | − | − | − |
| | | | (R350-354) | | PBMC | − | | | | | − | | | − | | − | + | + |
| | | | Group 10 | VAGINAL | TZM | − | NS | NS | NS | NS | − | | NS | − | | − | − | − |
| | | | (R355-359) | | PBMC | − | | | | | − | | | − | | − | + | ++ |
| | | Controls | Group 11 | IM | TZM | − | NS | NS | | NS | − | | NS | − | | − | − | − |
| | | | (R360-364) | | PBMC | − | | | | | + | | | − | | − | + | + |
| | | | Group 12 | NASAL | TZM | − | NS | NS | NS | NS | − | | NS | − | | − | − | − |
| | | | (R365-369) | | PBMC | − | | | | | + | | | − | | − | + | + |
| | | | Group 13 | VAGINAL | TZM | − | NS | NS | NS | NS | − | | NS | − | | − | − | − |
| | | | (R370-374) | | PBMC | − | | | | | + | | | − | | − | + | + |
| | | STNLS | Group 14 | SENTINELS | TZM | − | NS | NS | | NS | − | | NS | − | | − | | |
| | | | (R375-379) | | PBMC | + | | | | | − | | | − | | − | | |

KEYS:
NS: no sampling this day [symbol]
Negative result [symbol]
+for 1 or 2 rabbits [symbol]
+for 3 or 4 rabbits [symbol]
+for 5 rabbits [symbol]
Unclear result

The ELISA and neutralization assays indicated that FP-UGR7-MPR-A-2 (SEQ ID NO.: 1) and PR-A are the most potent antigens for induction of systemic and mucosal responses. However, the assays differ in ranking the most efficient routes of delivery. ELISA data indicates vaginal administration is most effective, followed by intramuscular and then nasal. The neutralizing assay indicates that vaginal and nasal routes of administration are equally effective, followed by the intramuscular route. In one study, ELISA data indicated that three nasal priming administrations followed by two IM boosts elicited a serum IgG response (only) in one rabbit and an IgA response in weck cels (vaginal samples) of all animals. Schedules with three or four IM administrations or three vaginal priming administations followed by an IM boost induced a positive serum IgG response in all rabbits and IgG and IgA responses in weck cels of all animals. By neutralizing assay, the three schedules tested exhibited a good neutralizing response in serum and in weck cels of all animals. Thus, the mucosal prime-IM boost approach and the IM-only schedules may be most suitable.

### B. Human Clinical Trial

The investigational product (HIV vaccine) for immunizing human beings may be the EN41-FPA2 suspension comprising the gp41-derived protein, FP-UGR7-MPR-A-2 (SEQ ID NO.: 1), formulated in liposomes containing monophosphoryl lipid A (MPLA), prepared as described above. Each mL of liposomal suspension contains 1 mg of FP-UGR7-MPR-A-2 and 800 μg of MPLA, and is stored at 5+/−3° C. The mode of administration will typically include at least one nasal administration followed by at least one intra-muscular (IM) administration (e.g., a prime-boost protocol). For example, three EN41-FPA2 priming immunisations may be administered by the nasal route followed by two EN41-FPA2 booster immunisations by the intramuscular route up to 28 days after the final nasal immunisation. The subject may be human beings (e.g., healthy female volunteers 18 to 55 years old at low risk of HIV infection). For nasal administration, one mL of EN41-FPA2 suspension may be mixed 1:1 (v/v) with HEC gel composition (4% w/w Natrosol HHX (Hydroxyethyl cellulose); 1.1% w/w Benzyl alcohol in PBS). One or more groups may receive 40 μL in a single nostril corresponding to 20 μg protein and 16 m MPLA. Another one or more groups may receive 200 μL in a single nostril corresponding to 100 μg of protein and 80 μg MPLA. And another one or more groups may receive 200 μL in each nostril, i.e. 400 μL corresponding to 200 μg of protein and 160 μg MPLA. The control nasal administration may include the HEC gel composition only. For IM administration, one mL of EN41-FPA2 suspension may be be mixed v/v with 0.9% NaCl (e.g., 400 μL of the diluted suspension, corresponding to 200 μg of protein and 160 μg MPLA). The control IM administration may include 0.9% NaCl only. An exemplary trial design is shown in Table 9:

TABLE 9
Treatment scheme: five groups of treatments, in three cohorts.

| Group | Treatment | Cohort 1 | Cohort 2 | Cohort 3 | Total |
|---|---|---|---|---|---|
| 1 | Nasal vaccine - | 4 | 0 | 0 | 4 |
| | Low-dose | | | | |
| | (20 μg in 40 μL) | | | | |
| | IM vaccine | | | | |
| | (200 μg in 400 μL) | | | | |
| 2 | Nasal vaccine - | 0 | 4 | 0 | 4 |
| | Mid-dose | | | | |
| | (100 μg in 200 μL) | | | | |
| | IM vaccine | | | | |
| | (200 μg in 400 μL) | | | | |
| 3 | Nasal vaccine - | 0 | 0 | 18 | 18 |
| | Full-dose | | | | |
| | (200 μg in 400 μL) | | | | |
| | IM vaccine | | | | |
| | (200 μg in 400 μL) | | | | |
| 4 | Nasal Placebo - | 0 | 0 | 18 | 18 |
| | 400 μL | | | | |
| | IM vaccine | | | | |
| | (200 μg in 400 μL) | | | | |
| 5 | Nasal placebo - | 2 | 2 | 0 | 4 |
| | 40 μL in | | | | |
| | Cohort 1/200 μL | | | | |
| | in Cohort 2 | | | | |
| | IM placebo | | | | |
| | (400 μL) | | | | |
| | Total | 6 | 6 | 36 | 48 |

The following parameters may be measured to determine the safety and immunogenicity of these systems (e.g., among many others as would be understood by those of ordinary skill in the art): EN41-FPA-2 specific serum IgG responses by ELISA assay induced by the vaccine candidate (e.g., up to 28 days after the final immunisation); the neutralising activity against HIV in serum and vaginal samples using PBMC and TZM-b1 assays (e.g., up to 28 days after the final immunisation); neutralising activity against HIV in serum and vaginal samples using PBMC and TZM-b1 assays (e.g., up to 6 months after the final immunisation); specific B cell responses (e.g., as measured by ELISPOT assay); inhibitory activity measured by virus capture assay; inhibitory activity by ADCC assay using primary NK cells; Fc-mediated inhibitory activity on macrophages; inhibition of HIV transfer from DC to CD4+ T lymphocytes (e.g., by antibodies); T-cell responses as determined using Intracellular Cytokine Staining (ICS) using multi-parametric flow cytometry or ELISPOT assay; kinetics of the immune response; inhibitory antibody activity (neutralization, ADCC, Fc-mediated inhibition) using, for example, purified IgG/IgA from plasma; the proportion of individuals mounting a serum IgG response to EN41-FPA2 at any time point up to 28 days (or 6 months) after the final immunisation with a three-fold increase from pre-immunisation baseline sample taken at visit 2 (week 0, priming #1) (if no serum sample is available at this time point, serum taken at visit 1, screening may be substituted); proportion of individuals mounting a serum IgA response to EN41-FPA2; proportion of individuals mounting a mucosal antibody response to EN41-FPA2 (either IgG or IgA) in vaginal secretion samples; increase from baseline of neutralising activity in serum, vaginal and/or cervico-vaginal secretions against Tier 1 virus, measured as IC80 in PBMC assays and IC50 in TZM-b1 assays (e.g., “baseline” being the result of the sample taken at visit 2, week 0 (or in the case of serum at screening visit 1, if no sample is available from visit 2)), where a positive response is represented by a three-fold increase from baseline occurring any time after the first immunisation; comparison of serum ELISA IgG titers in the various groups (e.g., groups 3 and 4 in Table 9) to investigate the importance of priming in the immunization scheme; proportion of volunteers with specific B cell responses measured by ELISPOT assay; proportion of volunteers with inhibitory activity measured by virus capture assay; proportion of volunteers with inhibitory activity by ADCC assay; proportion of volunteers with Fc-mediated inhibitory activity; proportion of volunteers with inhibition of HIV transfer from DC to CD4+ T lymphocytes; proportion of volunteers mounting a T cell response measured by either ICS or ELISPOT assay; kinetics assessed by measurement of the ELISA response at all timepoints. Adverse events (e.g., grade 3 or above) may also be monitored to confirm safety.

It is to be understood that any reference to a particular range includes all individual values and sub-ranges within that range as if each were individually listed herein. All references cited within this application are incorporated by reference in their entirety. While various embodiments may have been described in terms of being preferred, it is understood that variations and modifications will occur to those skilled in the art. Therefore, it is intended that the appended claims cover all such equivalent variations that come within the scope of this disclosure as claimed.

### REFERENCES

Ablation of the CDR H3 apex of the anti-HIV-1 broadly neutralizing antibody 2F5 abrogates neutralizing capacity without affecting core epitope binding. J. Virol. 2010 May; 84(9):4136-47
Agopian, et al. Secondary structure analysis of HIV-1-gp41 in solution and adsorbed to aluminum hydroxide by Fourier transform infrared spectroscopy. Biochim Biophys Acta. 2007 March; 1774(3):351-8. Epub 2007 January 3.
Aromatic residues at the edge of the antibody combining site facilitate viral glycoprotein recognition through membrane interactions. Proc Natl Acad Sci USA, 2010, 107:1529-34
Bianchi, et al. Vaccination with peptide mimetics of the gp41 prehairpin fusion intermediate yields neutralizing antisera against HIV-1 isolates. PNAS, June 2010, 107:10655-60
Caffrey et al. (2000 BBA) gp41 ectodomain is highly insoluble at physiological pH (Model structure PDB: 1IF3)
Caffrey et al. (2000) JBC The site for aggregation of gp41 ectodomain is located at the PID loop.
Fenyö, et al. International network for comparison of HIV neutralization assays: the NeutNet report. PLoS One. 2009; 4(2):e4505. Epub 2009 Feb. 20. (PBMC and Macrophages assays)
Holl, et al. Involvement of Fc gamma RI (CD64) in the mechanism of HIV-1 inhibition by polyclonal IgG purified from infected patients in cultured monocyte-derived macrophages. J. Immunol. 2004 Nov. 15; 173(10):6274-83 (PBMC and Macrophages assays)
Krell, et al. HIV-1 gp41 and gp160 are hyperthermostable proteins in a mesophilic environment. Characterization of gp41 mutants. Eur J. Biochem. 2004 April; 271(8):1566-79.
Li, et al. Human immunodeficiency virus type 1 env clones from acute and early subtype B infections for standardized assessments of vaccine-elicited neutralizing antibodies. J. Virol. 2005 August; 79(16):10108-25.
Peisajovich S G, Blank L, Epand R F, et al. On the interaction between gp41 and membranes: The immunodominant loop stabilizes gp41 helical hairpin conformation. J. Mol. Biol. 326, 1489-1501 (2003).
Relationship between Antibody 2F5 Neutralization of HIV-1 and Hydrophobicity of Its Heavy Chain Third Complementarity-Determining Region. J. Virol., 2010, 84: 2955-62
Role of HIV membrane in neutralization by two broadly neutralizing antibodies. Proc Natl Acad Sci USA, 2010, 106:20234-9
Umrethial, et al. P24 gp41 sublingual or vaginal delivery strategies for mucosal immunization. (Poster presented on a Europrise meeting, 2009 in Budapest)

1. An isolated gp41 polypeptide modified to exhibit at least one characteristic different from a wild-type gp41 polypeptide, the at least one characteristic being selected from the group consisting of reduced hydrophobicity, increased solubility at physiological pH, increased net charge, and decreased propensity to form a post-fusion conformation.

2. The isolated gp41 polypeptide of claim 1 comprising at least one amino acid substitution selected from the group consisting of leucine 81 (L81), tryptophan 85 (W85), threonine 95 (T95), alanine 96 (A96), and an equivalent thereof, or a polypeptide having 80-99% identity thereto.

3. The isolated gp41 polypeptide of claim 1 wherein the substitution is made to SEQ ID NO.:10.

4. (canceled)

5. The isolated polypeptide of claim 2 wherein the substitution of L81 is by aspartic acid (D), the substitution of W85 is by glutamic acid (E), the substitution of T95 is by proline (P), the substitution of A96 is by glutamic acid (E), and equivalents thereof.

6. The isolated gp41 polypeptide of claim 1 comprising a first amino acid substitution selected from the group consisting of leucine 81 (L81), tryptophan 85 (W85), threonine 95 (T95), alanine 96 (A96), and equivalents thereof and a second amino acid substitution selected from the group consisting of leucine 91 (L91), isoleucine 92 (I92), tryptophan 103 (W103), and equivalents thereof.

7-8. (canceled)

9. The isolated polypeptide of claim 1 wherein the amino acid substitutions are at leucine 81 (L81), tryptophan 85 (W85), threonine 95 (T95), alanine 96 (A96), leucine 91 (L91), isoleucine 92 (I92), tryptophan 103 (W103), and equivalents thereof.

10. The isolated polypeptide of claim 9 wherein the substitution of L81 or equivalent thereof is by aspartic acid (D), the substitution of W85 or equivalent thereof is by glutamic acid (E), the substitution of L91 or equivalent thereof is by glycine, the substitution of 192 or equivalent thereof is by aspartic acid (D), the substitution of T95 or equivalent thereof is by proline (P), the substitution of A96 or equivalent thereof is by glutamic acid (E), the substitution of W103 or equivalent thereof is by aspartic acid (D).

11. The isolated polypeptide of claim 1 further comprising a deletion of the polar region.

12. The isolated polypeptide of claim 11 wherein the polar region is AGSTMGARSMTLTVQA (SEQ ID NO.: 3).

13. An isolated polypeptide comprising SEQ ID NO.: 1.

14-15. (canceled)

16. The isolated polypeptide of claim 13 further comprising the amino acid sequence MHKVHGSGSGS (SEQ ID NO.: 13).

17. The polypeptide of claim 1 in trimeric form.

18. A nucleic acid encoding the isolated polypeptide of claim 1.

19-23. (canceled)

24. A composition comprising the isolated polypeptide of claim 1 and a pharmaceutically acceptable carrier.

25. The composition of claim 24 further comprising an adjuvant.

26-27. (canceled)

28. A composition comprising a liposome comprising the isolated polypeptide of claim 1.

29. A composition of claim 28 in the form of a liposome.

30-32. (canceled)

33. A method for producing a liposome of claim 28, the method comprising combining a lipid with the polypeptide in the presence of Tween 20 and isolating the liposome.

34-36. (canceled)

37. A method of eliciting an immune response in a mammal, the method comprising administering to the mammal a composition comprising the isolated polypeptide of claims 1.

38. An immunogenic composition comprising the isolated polypeptide of claim 1.

39. A vaccine composition comprising the isolated polypeptide of claim 1.

40. A method of eliciting an immune response in a mammal, the method comprising administering to the mammal a composition of 38.

41. An isolated antibody reactive with the polypeptide of claim 1.

42. (canceled)

43. A host cell comprising the nucleic acid of claim 18.

44. A method for producing an isolated polypeptide from a host cell of claim 43 comprising expressing the polypeptide in a host cell and isolating the polypeptide.

45. A method for producing an immunogenic liposome, the method comprising:
combining an ethanolic lipid solution, a micellar protein solution comprising a polypeptide of claim 1 and a detergent, and a buffer;
precipitating the lipid components in the aqueous phase; and,
removing residual detergent.

46-52. (canceled)

53. The polypeptide of claim 1 comprising the amino acid sequence:
(SEQ ID NO.: 12)
YIKIFIMIVGGLVGLRIVFAVLSIVNRVRQGYSPLSFQTHLPTPRGPDRP
EGIEEEGGERDRDRSIRLVNGSLALIWDDLRSLCLFSYHRLRDLLLIVTR
IVELLGRRGWEALKYWWNLLQYWSQELKNSAVSLLNATAIAVAEGTDRVI
EVVQGACRAIRHIPRRIRQGLERILL.
